(19) United States
(12) Patent Application Publication
(10) Pub. No.: US 2025/0375366 A1
(43) Pub. Date: Dec. 11, 2025

(54) PROCESS FOR TREATING KERATIN FIBRES USING A CARNITINE SALT OR A CARNITINE DERIVATIVE

(71) Applicant: L'OREAL, Paris (FR)

(72) Inventors: Nawel BAGHDADLI, AULNAY-SOUS-BOIS (FR); Andrew GREAVES, AULNAY-SOUS-BOIS (FR)

(21) Appl. No.: 18/876,579

(22) PCT Filed: Jun. 21, 2023
(86) PCT No.: PCT/EP2023/066773

§ 371 Date: Dec. 18, 2024

(30) Foreign Application Priority Data
Jun. 22, 2022 (FR) FR2206166

(51) Int. Cl. A61K 8/44; A61K 8/362; A61K 8/46; A61K 8/55; A61Q 5/00

(52) U.S. Cl. CPC A61K 8/44; A61K 8/362; A61K 8/466; A61K 8/55; A61Q 5/002

(57) ABSTRACT

The present invention relates to a process for treating keratin fibres comprising the application to the fibres of a composition comprising at least one chemical oxidizing agent, at least one (bi)carbonate and at least one silicate, the process also using at least one carnitine salt or carnitine derivative.

### TECHNICAL FIELD OF THE INVENTION

The present invention relates to a process for treating keratin fibres comprising the application to the fibres of a composition comprising at least one chemical oxidizing agent, at least one (bi)carbonate and at least one silicate, the process also using at least one carnitine salt or carnitine derivative.

### BACKGROUND OF THE INVENTION

When a person wishes to change their hair colour, in particular when they wish to obtain a lighter colour than their original colour, it is often necessary to carry out, beforehand, hair lightening or bleaching. To do this, lightening or bleaching products are used.

The lightening of the hair is evaluated by the “tone depth”, which characterizes the degree or level of lightening. The notion of “tone” is based on the classification of natural shades, one tone separating each shade from the shade immediately following or preceding it. This definition and the classification of natural shades are well known to hairstyling professionals and are published in the book Sciences des traitements capillaires [The Science of Hair Care] by Charles Zviak, 1988, published by Masson, pp. 215 and 278. The tone depths range from 1 (black) to 10 (very light blond), one unit corresponding to one tone; the higher the figure, the lighter the shade.

It is known practice to lighten or bleach hair with lightening or bleaching compositions containing at least one chemical oxidizing agent, under alkaline pH conditions in the vast majority of cases. The role of this oxidizing agent is to degrade the melanin of hair, which, depending on the nature of the oxidizing agent present and on the pH conditions, results in more or less pronounced lightening of the fibres. Thus, for relatively mild lightening, the oxidizing agent is generally hydrogen peroxide. When greater lightening is desired, in particular when the treated hair is dark, use is normally made of persulfates in the presence of hydrogen peroxide. However, the lightening obtained by the action of such a combination is not always satisfactory because hair with rather unattractive orangey-yellow shades which are very different from natural shades is obtained, thereby complicating the subsequent colouring by limiting it to the obtaining of warm tones. Furthermore, persulfate-based lightening compositions can lead to a degradation of the quality and integrity of the fibre. The hair may then end up deformed or damaged, which notably leads to significant breakage during combing or disentangling.

To overcome these disadvantages, then, it is common practice to employ haircare compositions intended for conditioning the hair by giving it satisfactory cosmetic properties, notably smoothness, sheen, a soft feel, suppleness and lightness, and also good disentangling properties leading to easy combing and good manageability of the hair, which is thus easier to style and holds its shape. However, the conditioning effect obtained via these haircare compositions fades out rapidly over time and does not allow the hair cortex to be strengthened, notably by improving the density of linkages between the proteins present in the hair cortex, in order to protect or repair the hair and to reduce the breakage thereof, for example during combing or disentangling. Furthermore, these care compositions may, in certain cases, impair the dyeing or bleaching of the hair thus treated.

There is thus a real need to develop a process which makes it possible to obtain efficient lightening of keratin fibres, in particular of dark keratin fibres, with a less yellow and more natural result. Such a process will also have to be more attentive to the quality and the integrity of the fibres and make it possible in particular to maintain, or even improve, the quality and integrity of the keratin fibres and in particular to reduce the breakage thereof, while avoiding impairing the lightening, notably the deyellowing, of the fibres thus treated.

The applicant has discovered, surprisingly, that all or some of these objectives can be achieved by means of the process according to the present invention.

### SUMMARY OF THE INVENTION

According to a first aspect, a subject of the present invention is a process for treating keratin fibres, comprising
a) applying to the keratin fibres one or more compounds D chosen from the compounds of formula (I), solvates thereof, and mixtures thereof:

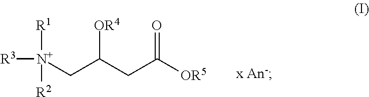

in which formula (I):
   R¹, R² and R³, which may be identical or different, represent a (hydroxy)(C₁-C₄)alkyl group;
   R⁴ represents a hydrogen atom, a (C₁-C₄)alkyl group or a group —C(O)R, in which R is a (C₁-C₃)alkyl group;
   R⁵ represents a hydrogen atom, a (C₁-C₄)alkyl group, an alkali metal or an alkaline-earth metal;
   An⁻ represents an organic anion;
   x is a stoichiometric coefficient chosen so as to ensure the electrical neutrality of the compounds of formula (I);
b) applying to the keratin fibres a composition (B) comprising:
i) one or more chemical oxidizing agents chosen from hydrogen peroxide, hydrogen peroxide-generating systems other than peroxygenated salts, and mixtures thereof;
ii) one or more compounds chosen from carbonates, carbonate-generating systems, bicarbonates, bicarbonate-generating systems and mixtures thereof, preferably from carbonates, bicarbonates and mixtures thereof;
iii) one or more silicates;
in which the compound(s) D are:
   in the composition (B); and/or
   in a separate composition (A), composition (A) being applied to the keratin fibres before composition (B); and/or
   in a separate composition (C), composition (C) being applied to the keratin fibres after composition (B);
it being understood that the compositions (A) and (C) may be identical or different.

According to a second aspect, a subject of the present invention is a composition (B) as defined previously comprising one or more compounds D as defined previously.

According to a third aspect, a subject of the present invention is the use of the composition(s) (A) and/or (C) as defined previously for reducing the damage to keratin fibres treated by a process for treating keratin fibres using composition (B) as defined previously.

According to a fourth aspect, a subject of the present invention is a multicompartment device comprising:
a first compartment containing a composition (B1) comprising:
   one or more chemical oxidizing agents chosen from hydrogen peroxide, hydrogen peroxide-generating systems other than peroxygenated salts, and mixtures thereof; and
a second compartment containing a composition (B2) comprising:
   one or more compounds chosen from carbonates, carbonate-generating systems, bicarbonates, bicarbonate-generating systems and mixtures thereof; and
   one or more silicates; and
   optionally one or more colouring agents, preferably chosen from direct dyes, oxidation dyes and mixtures thereof; and
a third compartment containing a composition (A) or (C) as defined previously, it being possible for composition (A) or (C) to optionally comprise one or more colouring agents, preferably chosen from direct dyes, oxidation dyes and mixtures thereof; and
optionally a fourth compartment containing a composition (B3) comprising one or more colouring agents, preferably chosen from direct dyes, oxidation dyes and mixtures thereof;
   it being understood that at least one of compositions (B1), (B2), (B3), (A) or (C) comprises one or more compounds D as defined previously.

### DETAILED DESCRIPTION OF THE INVENTION

For the purposes of the present invention and unless otherwise indicated:
the term “keratin fibres” means fibres of human or animal origin, such as head hair, bodily hairs, the eyelashes, the eyebrows, wool, angora, cashmere or fur. According to the present invention, the keratin fibres are preferably human keratin fibres, more preferentially the hair.
The term “salt” means an addition salt with an organic or mineral acid or base. The addition salts with an acid are notably chosen from the addition salts with acids such as hydrochloric acid, hydrobromic acid, sulfuric acid, citric acid, succinic acid, tartaric acid, lactic acid, tosylic acid, benzenesulfonic acid, phosphoric acid or acetic acid. The addition salts with a base are notably chosen from the addition salts with bases such as the basifying agents defined below, notably alkali metal hydroxides, alkaline-earth metal hydroxides, aqueous ammonia, amines or alkanolamines.
The term “alkyl group” means a saturated, linear or branched, hydrocarbon radical.
The term “(Cₓ-C_{y})alkyl group” means an alkyl group comprising from x to y carbon atoms, preferably methyl, ethyl, n-propyl, isopropyl or butyl, more preferentially methyl or n-propyl, even more preferentially methyl.
The term “alkylene group” means a linear or branched divalent saturated C₁-C₈, in particular C₁-C₆, preferably C₁-C₄ hydrocarbon-based radical such as methylene, ethylene or propylene.
The term “hydroxy(Cₓ-C_{y})alkyl group” means a (Cₓ-C_{y})alkyl group, at least one of the hydrogen atoms of which is replaced with a hydroxyl (—OH) group.
The term “alkoxy group” means an alkyl group bonded to an oxygen atom.
The term “(Cₓ-C_{y})alkoxy group” means an alkoxy group comprising from x to y carbon atoms.
The term “hydroxy(Cₓ-C_{y})alkoxy group” means a (Cₓ-C_{y})alkoxy group, at least one of the hydrogen atoms of which is replaced with a hydroxyl (—OH) group.
The term “(hydroxy)(Cₓ-C_{y})alkyl group” means a hydroxy(Cₓ-C_{y})alkyl group or a (Cₓ-C_{y})alkyl group.
The term “(hydroxy)(Cₓ-C_{y})alkoxy group” means a hydroxy(Cₓ-C_{y})alkoxy group or a (Cₓ-C_{y})alkoxy group.
The term “(hetero)cycle” means a heterocycle or a carbocycle.
The term “heterocycle” means a 4- to 8-membered, preferably 5- or 6-membered, saturated or unsaturated or aromatic cyclic group, the ring members of which consist of carbon atoms and of at least one heteroatom chosen from: O, S and N, preferably O and N. A heterocycle denotes, for example, morpholinyl, piperidyl, tetrahydrofuryl or pyrrolidinyl.
The term “carbocycle” means a 4- to 8-membered, preferably 5- or 6-membered, saturated or unsaturated cyclic group, the ring members of which consist of carbon atoms. A carbocycle denotes, for example, a cyclopentyl or cyclohexyl ring.
The term “aryl” means a fused or nonfused monocyclic or polycyclic carbon-based group comprising from 6 to 22 carbon atoms, and in which at least one ring is aromatic. Preferably, the aryl radical is a phenyl, biphenyl, naphthyl, anthracenyl or tetrahydronaphthyl, and more preferentially a phenyl.

The “aryl” or “heterocycle” or “carbocycle” group or the aryl or heterocyclic or carbocyclic part of a group may be substituted with at least one substituent borne by a carbon atom, chosen from:
a (C₁-C₆)alkyl group, preferably a (C₁-C₄)alkyl group, which is optionally substituted with one or more groups chosen from hydroxyl and (C₁-C₂)alkoxy;
a halogen atom such as chlorine;
a hydroxyl or thiol group;
a (C₁-C₆)alkoxy group or a (C₁-C₆)alkylthio group;
a (C₂-C₄)(poly)hydroxyalkoxy group.

A (hetero)cyclic group or a nonaromatic part of an aryl group may also be substituted with one or more oxo groups.
The term “protecting group for a phenol function” means a protecting group such as those described in Greene's Protective Groups in Organic Synthesis, P. G. M. Wutz, T. Greene, published by Wiley-Blackwell, preferably an acyl group, in particular an acetyl group.
The term “silicate” means a silicic acid salt.
The term “colouring agent” means an oxidation dye, a direct dye or a pigment.
The term “oxidation dye” means an oxidation dye precursor chosen from oxidation bases and couplers. Oxidation bases and couplers are colourless or sparingly coloured compounds, which, via a condensation reaction in the presence of an oxidizing agent, give a coloured species.
The term “direct dye” means a natural and/or synthetic dye, including in the form of extract(s), other than oxidation dyes. These are coloured compounds that will spread superficially on the fibre. They may be ionic or nonionic, i.e. anionic, cationic, neutral or nonionic.
The term “chemical oxidizing agent” means an oxidizing agent other than atmospheric oxygen.

Unless otherwise indicated, when compounds are mentioned in the present patent application, this also includes the optical isomers thereof, the geometric isomers thereof, the tautomers thereof, the salts thereof or the solvates thereof such as hydrates, and mixtures thereof.

The terms “at least one” and “one or more” are synonymous and may be used interchangeably.

The expressions “lightening” and “bleaching” are synonymous and can be used interchangeably.

### Process

According to a first aspect, a subject of the present invention is a process for treating keratin fibres as defined previously.

According to a preferred embodiment, the process according to the present invention is a process for lightening keratin fibres.

The applicant has noted, surprisingly, that the process according to the present invention makes it possible to obtain efficient lightening of keratin fibres with a less yellow and more natural result.

When the colour of the keratin fibres treated by the process according to the invention is compared to the colour of the keratin fibres treated by processes using lightening compositions known from the prior art, values of b* measured in the CIE L*a*b* system that are lower for the process according to the invention than for the processes using lightening compositions known from the prior art, at equivalent intensity level L*, are observed.

Furthermore, the process according to the present invention is more attentive to the quality of the fibres and makes it possible in particular to maintain, or even improve, the quality of the keratin fibres and in particular to reduce the breakage thereof, while avoiding impairing the lightening, notably the deyellowing, of the fibres thus treated. According to a preferred embodiment, the compound(s) D are in composition (B). According to another preferred embodiment, the compound(s) D are in a separate composition (A), composition (A) being applied to the keratin fibres before composition (B).

According to another preferred embodiment, the compound(s) D are in a separate composition (C), composition (C) being applied to the keratin fibres after composition (B).

The combinations of these various embodiments are also conceivable.

### Compounds D

The compound(s) D are present in composition (A) in a total content by weight preferably ranging from 1% to 25% by weight, more preferentially ranging from 5% to 20% by weight, relative to the total weight of composition (A).

The compound(s) D are present in composition (B) in a total content by weight preferably ranging from 1% to 20% by weight, more preferentially ranging from 2% to 15% by weight, even more preferentially ranging from 2% to 12% by weight, relative to the total weight of composition (B).

The compound(s) D are present in composition (C) preferably in a total content by weight ranging preferably from 1% to 25% by weight, more preferentially ranging from 2% to 20%, even more preferentially ranging from 2% to 15% by weight relative to the total weight of composition (C).

The cationic portion of the compounds of formula (I) is carnitine or a derivative thereof and the anionic portion An⁻ of the compounds of formula (I) is an organic anion.

### Anionic Portion

Preferably, An⁻ represents an organic anion chosen from:
the compounds of formula ⁻O—C(O)-ALK—C(O)—OM (IIa),

in which:
ALK represents a (C₁-C₆)alkylene group optionally substituted with one or more hydroxyl (—OH) groups, preferably a (C₂-C₄)alkylene group;
M represents a hydrogen atom, a negative charge, an alkali metal or an alkaline-earth metal, preferably a negative charge or a hydrogen atom;
the compounds of formula (IIb), (IIIb) or (IVb) below:

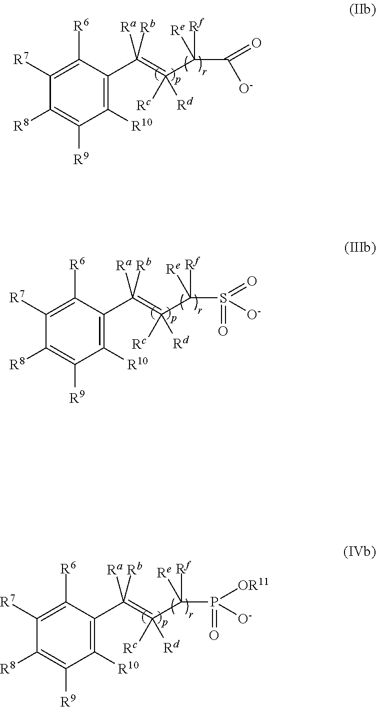

in which:
R⁶ to R¹⁰ represent, independently of each other:
   a hydrogen atom;
   a (C₁-C₆)alkyl or (C₁-C₆)alkoxy group, the (C₁-C₆)alkyl or (C₁-C₆)alkoxy group being optionally substituted with one or more identical or different groups chosen from: —OR, —NR′R″, —C(O)OR, —O—C(O)—R, aryl and —CF₃ and/or optionally interrupted with one or more groups or heteroatoms chosen from: O, NR′, CO, S, SO and SO₂; R representing a hydrogen atom or a (C₁-C₄)alkyl group, R′ and R″ representing, independently of each other, a hydrogen atom, a (C₁-C₄)alkyl group or a (C₁-C₄)alkylcarbonyl group such as acetyl, it being understood that the group —NR′R″ is other than an —NH₂ group;
   a halogen atom;
   a group —O—X, in which X represents a hydrogen atom or a protecting group for the phenol function such as an acyl group, in particular an acetyl group;

it being understood that two adjacent groups R⁶ to R¹⁰ may also form, together with the carbon atoms that bear them, a saturated or unsaturated or aromatic 5- or 6-membered (hetero)cycle;
R^{a}, R^{b}, R^{c}, R^{d}, R^{e} and R^{f} represent, independently of each other:
   a hydrogen atom;
   a (C₁-C₆)alkyl group optionally substituted with one or more identical or different groups chosen from: —OR, —NR′R″, —C(O)OR, —O—C(O)—R, (hetero)cycle, —C(O)—R, —NH—C(O)—R, —C(O)—NH—R, with R, R′ and R″ as defined previously, it being understood that the group —NR′R″ is other than an —NH₂ group;
   a (C₁-C₆)alkoxy group optionally substituted with one or more identical or different groups chosen from: —OR, —NR′R″, —C(O)OR, —O—C(O)—R, aryl and —CF₃ and/or optionally interrupted with one or more groups or heteroatoms chosen from: O, NR′, CO, S, SO and SO₂, with R, R′ and R″ as defined previously, it being understood that the group —NR′R″ is other than an —NH₂ group;
   a halogen atom;
   a group —O—X with X as defined previously;

it being understood that:
(R^{a} and R^{b}) and/or (R^{c} and R^{d}) may form, together with the carbon atom which bears them, a C═O bond;
R^{a} and R^{b} may form, together with the carbon atom which bears them, a saturated or unsaturated, 4- to 8-membered, in particular 4- to 6-membered, (hetero)cycle, and more particularly a 5- or 6-membered carbocycle;
R^{a} or R^{b} or R^{c} or R^{d} may form with R⁶ or R¹⁰ and the carbon atoms that bear them a 4- to 8-membered (hetero)cycle, more particularly a 5- to 6-membered carbocycle;
[symbol] represents a single bond or an ethylenic double bond, it being understood that when [symbol] represents an ethylenic double bond, R^{b} and R^{c} are absent;
p is an integer equal to 0 or 1;
r is an integer equal to 0 or 1;
R¹¹ represents a hydrogen atom, a negative charge, an alkali metal or an alkaline-earth metal, preferably a negative charge;
   the compounds of formula (IIc) below:

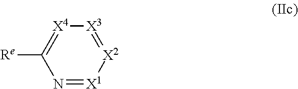

in which:
X¹, X², X³ and X⁴ represent, independently of each other, a nitrogen atom or CR⁷, it being understood that at least two from among X¹, X², X³ and X⁴ represent CR⁷;
R⁶ and the substituents R⁷ represent, independently of each other, a hydrogen atom or a group chosen from:
   a group of formula (a) or (b) or (c) below:

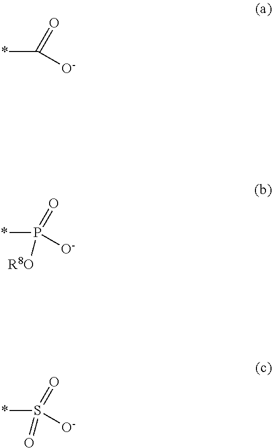

in which formula (b) R⁸ represents a hydrogen atom, a negative charge, an alkali metal or an alkaline-earth metal, preferably a negative charge;
a hydroxyl group;
a thiol group or SR′;
a (hydroxy)(C₁-C₄)alkoxy group;
a group —CO₂R′;
a group —OCOR′;
a (C₁-C₄)alkyl group optionally substituted with one or more identical or different groups chosen from —CO₂R′, —OR′, —OH, —SH, —SR′, —NRₐR_{b}, —CO—NR′ₐR′_{b} and/or optionally interrupted with one or more non-adjacent oxygen atoms;
a halogen atom;
a —CF3 group;
a group R′—CO—NR′ₐ—;
a group —NRₐR_{b};
a group R′ₐR′_{b}N—CO—;
a cyano group;

in which:
R′ represents a (C₁-C₄)alkyl group; and
Rₐ and R_{b} represent, independently of each other, a hydrogen atom or a (C₁-C₄)alkyl group, it being understood that at least one from among Rₐ and R_{b} is other than H; and
R′ₐ and R′_{b} represent, independently of each other, a hydrogen atom or a (hydroxy)(C₁-C₄)alkyl group, preferably a hydrogen atom or a (C₁-C₄)alkyl group;

it being understood that:
Rₐ and R_{b} may form, together with the nitrogen atom that bears them, a 5- or 6-membered heterocycle;
when X² and X³ each represent a group CR⁷, the two substituents R⁷ may form, together with the carbon atoms that bear them, a 5- or 6-membered (hetero)cycle;
R⁶ and/or at least one of the substituents R⁷ represent a group of formula (a) or (b) or (c), preferably a group of formula (a);
and mixtures thereof.

### Compounds of Formula (IIa)

The compounds of formula (IIa) are preferably chosen from the compounds of formula (IIa₁), (IIa₂), (IIa₃), (IIa₄), (IIa₅) or (IIa₆) below:

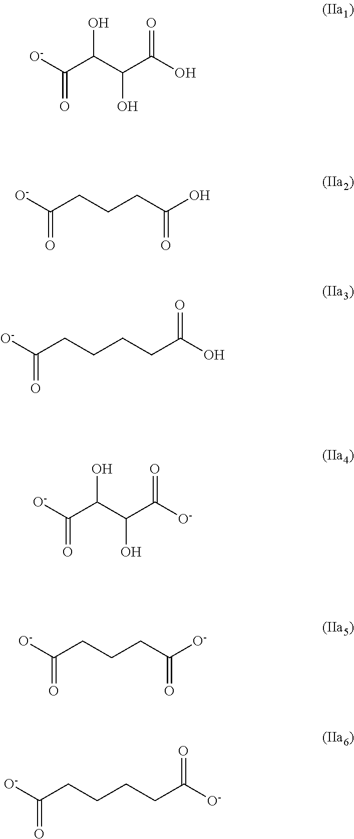 [SMILES: O=C([O-])C(O)C(O)C(=O)O ; O=C([O-])CCCC(=O)O ; O=C([O-])CCCCC(=O)O ; O=C([O-])C(O)C(O)C(=O)[O-] ; O=C([O-])CCCC(=O)[O-] ; O=C([O-])CCCCC(=O)[O-]]

### Compounds of Formula (IIb), (IIIb) or (IVb)

### First Variant

According to a first variant, the compounds of formula (IIb), (IIIb) or (IVb) are respectively chosen from the compounds of formula (IIIb₁), (IIIb₁) or (IVb₁) below:

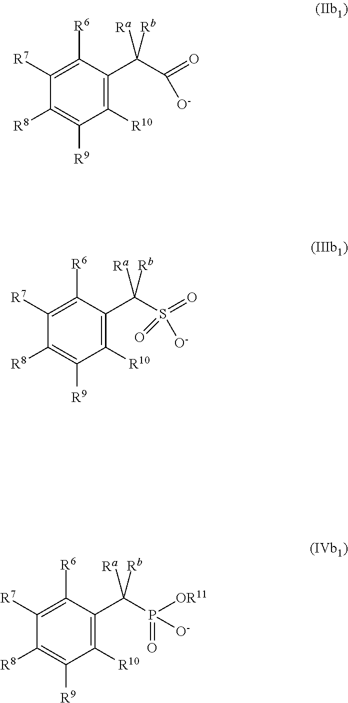

in which:
R⁶ to R¹⁰ represent, independently of each other:
   a hydrogen atom;
   a (C₁-C₆)alkyl or (C₁-C₆)alkoxy group, the (C₁-C₆)alkyl or (C₁-C₆)alkoxy group being optionally substituted with one or more identical or different groups chosen from: —OR, —NR′R″, —C(O)OR, —O—C(O)—R, aryl and —CF₃ and/or optionally interrupted with one or more groups or heteroatoms chosen from: O, NR′, CO, S, SO and SO₂; R representing a hydrogen atom or a (C₁-C₄)alkyl group, R′ and R″ representing, independently of each other, a hydrogen atom, a (C₁-C₄)alkyl group or a (C₁-C₄)alkylcarbonyl group such as acetyl, it being understood that the group —NR′R″ is other than an —NH₂ group;
   a halogen atom;
   a group —O—X, in which X represents a hydrogen atom or a protecting group for the phenol function such as an acyl group, in particular an acetyl group;

it being understood that two adjacent groups R⁶ to R¹⁰ may also form, together with the carbon atoms that bear them, a saturated or unsaturated or aromatic 5- or 6-membered (hetero)cycle;
R^{a} and R^{b} represent, independently of each other:
   a hydrogen atom;
   a (C₁-C₆)alkyl group optionally substituted with one or more identical or different groups chosen from: —OR, —NR′R″, —C(O)OR, —O—C(O)—R, (hetero)cycle, —C(O)—R, —NH—C(O)—R, —C(O)—NH—R, with R, R′ and R″ as defined previously, it being understood that the group —NR′R″ is other than an —NH₂ group;
   a (C₁-C₆)alkoxy group optionally substituted with one or more identical or different groups chosen from: —OR, —NR′R″, —C(O)OR, —O—C(O)—R, aryl and —CF₃ and/or optionally interrupted with one or more groups or heteroatoms chosen from: O, NR′, CO, S, SO and SO₂, with R, R′ and R″ as defined previously, it being understood that the group —NR′R″ is other than an —NH₂ group;
   a halogen atom;
   a group —O—X with X as defined previously;

it being understood that:
R^{a} and R^{b} may form, together with the carbon atom which bears them, a C═O bond;
R^{a} and R^{b} may form, together with the carbon atom which bears them, a saturated or unsaturated, 4- to 8-membered, in particular 4- to 6-membered, (hetero)cycle, and more particularly a 5- or 6-membered carbocycle;
R^{a} or R^{b} may form with R⁶ or R¹⁰ and the carbon atoms that bear them a 4- to 8-membered (hetero)cycle, more particularly a 5- to 6-membered carbocycle.
R¹¹ represents a hydrogen atom, a negative charge, an alkali metal or an alkaline-earth metal, preferably a negative charge.

Preferably, according to this first variant, R⁶ to R¹⁰ represent, independently of each other:
a hydrogen atom;
a (C₁-C₄)alkyl group;
a (C₁-C₄)alkoxy group;
a hydroxy(C₁-C₄)alkyl group;
a halogen atom;
a hydroxyl group;
an —O—C(O)—CH₃ group;

it being understood that two adjacent groups R⁶ to R¹⁰ may also form, together with the carbon atoms that bear them, a saturated 5- or 6-membered (hetero)cycle.

More preferentially, according to this first variant, R⁶ to R¹⁰ represent, independently of each other:
a hydrogen atom;
a (C₁-C₄)alkyl group;
a (C₁-C₄)alkoxy group;
a hydroxy(C₁-C₄)alkyl group;
a hydroxyl group.

Preferably, according to this first variant, R^{a} and R^{b} represent, independently of each other:
a hydrogen atom;
a (C₁-C₄)alkyl group;
a hydroxy(C₁-C₄)alkyl group;
a (C₁-C₄)alkoxy group;
a halogen atom;
a hydroxyl group;
an —O—C(O)—CH₃ group;

it being understood that:
R^{a} and R^{b} may form, together with the carbon atom which bears them, a C═O bond;
R^{a} or R^{b} may form with R⁶ or R¹⁰ and the carbon atoms that bear them a 4- to 8-membered (hetero)cycle, more particularly a 5- to 6-membered carbocycle.

More preferentially, according to this first variant, R^{a} and R^{b} represent, independently of each other:
a hydrogen atom;
a (C₁-C₄)alkyl group;
a (C₁-C₄)alkoxy group;
a hydroxy(C₁-C₄)alkyl group;
a hydroxyl group.

### Second Variant

According to a second variant, the compounds of formula (IIb), (IIIb) or (IVb) are respectively chosen from the compounds of formula (IIb₂), (IIIb₂) or (IVb₂) below:

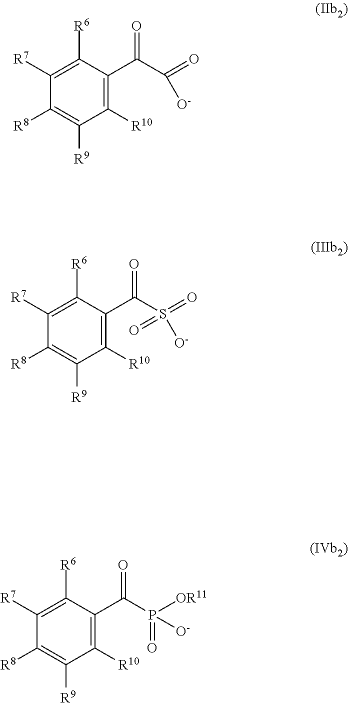

in which:
R⁶ to R¹⁰ represent, independently of each other:
   a hydrogen atom;
   a (C₁-C₆)alkyl or (C₁-C₆)alkoxy group, the (C₁-C₆)alkyl or (C₁-C₆)alkoxy group being optionally substituted with one or more identical or different groups chosen from: —OR, —NR′R″, —C(O)OR, —O—C(O)—R, aryl and —CF₃ and/or optionally interrupted with one or more groups or heteroatoms chosen from: O, NR′, CO, S, SO and SO₂; R representing a hydrogen atom or a (C₁-C₄)alkyl group, R′ and R″ representing, independently of each other, a hydrogen atom, a (C₁-C₄)alkyl group or a (C₁-C₄)alkylcarbonyl group such as acetyl, it being understood that the group —NR′R″ is other than an —NH₂ group;
   a halogen atom;
   a group —O—X, in which X represents a hydrogen atom or a protecting group for the phenol function such as an acyl group, in particular an acetyl group;

it being understood that two adjacent groups R⁶ to R¹⁰ may also form, together with the carbon atoms that bear them, a saturated or unsaturated or aromatic 5- or 6-membered (hetero)cycle;
R¹¹ represents a hydrogen atom, a negative charge, an alkali metal or an alkaline-earth metal, preferably a negative charge.

Preferably, according to this second variant, R⁶ to R¹⁰ represent, independently of each other:
a hydrogen atom;
a (C₁-C₄)alkyl group;
a (C₁-C₄)alkoxy group;
a hydroxy(C₁-C₄)alkyl group;
a halogen atom;
a hydroxyl group;
an —O—C(O)—CH₃ group;

it being understood that two adjacent groups R⁶ to R¹⁰ may also form, together with the carbon atoms that bear them, a saturated 5- or 6-membered (hetero)cycle.

More preferentially, according to this second variant, R⁶ to R¹⁰ represent, independently of each other:
a hydrogen atom;
a (C₁-C₄)alkyl group;
a (C₁-C₄)alkoxy group;
a hydroxy(C₁-C₄)alkyl group;
a hydroxyl group.

### Third Variant

According to a third variant, the compounds of formula (IIb), (IIIb) or (IVb) are respectively chosen from the compounds of formula (IIb₃), (IIIb₃) or (IVb₃) below:

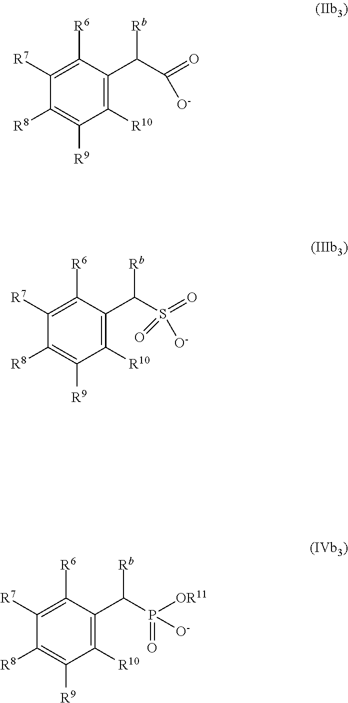

in which:
R⁶ to R¹⁰ represent, independently of each other:
   a hydrogen atom;
   a (C₁-C₆)alkyl or (C₁-C₆)alkoxy group, the (C₁-C₆)alkyl or (C₁-C₆)alkoxy group being optionally substituted with one or more identical or different groups chosen from: —OR, —NR′R″, —C(O)OR, —O—C(O)—R, aryl and —CF₃ and/or optionally interrupted with one or more groups or heteroatoms chosen from: O, NR′, CO, S, SO and SO₂; R representing a hydrogen atom or a (C₁-C₄)alkyl group, R′ and R″ representing, independently of each other, a hydrogen atom, a (C₁-C₄)alkyl group or a (C₁-C₄)alkylcarbonyl group such as acetyl, it being understood that the group —NR′R″ is other than an —NH₂ group;
   a halogen atom;
   a group —O—X, in which X represents a hydrogen atom or a protecting group for the phenol function such as an acyl group, in particular an acetyl group;

it being understood that two adjacent groups R⁶ to R¹⁰ may also form, together with the carbon atoms that bear them, a saturated or unsaturated or aromatic 5- or 6-membered (hetero)cycle;
R^{b} represents:
   a hydrogen atom;
   a (C₁-C₆)alkyl group optionally substituted with one or more identical or different groups chosen from: —OR, —NR′R″, —C(O)OR, —O—C(O)—R, (hetero)cycle, —C(O)—R, —NH—C(O)—R, —C(O)—NH—R, with R, R′ and R″ as defined previously, it being understood that the group —NR′R″ is other than an —NH₂ group;
   a (C₁-C₆)alkoxy group optionally substituted with one or more identical or different groups chosen from: —OR, —NR′R″, —C(O)OR, —O—C(O)—R, aryl and —CF₃ and/or optionally interrupted with one or more groups or heteroatoms chosen from: O, NR′, CO, S, SO and SO₂, with R, R′ and R″ as defined previously, it being understood that the group —NR′R″ is other than an —NH₂ group;
   a halogen atom;
   a group —O—X with X as defined previously;

it being understood that:
R^{b} may form with R⁶ or R¹⁰ and the carbon atoms that bear them a 4- to 8-membered (hetero)cycle, more particularly a 5- to 6-membered carbocycle.
R¹¹ represents a hydrogen atom, a negative charge, an alkali metal or an alkaline-earth metal, preferably a negative charge.

Preferably, according to this third variant, R⁶ to R¹⁰ represent, independently of each other:
a hydrogen atom;
a (C₁-C₄)alkyl group;
a (C₁-C₄)alkoxy group;
a hydroxy(C₁-C₄)alkyl group;
a halogen atom;
a hydroxyl group;
an —O—C(O)—CH₃ group;

it being understood that two adjacent groups R⁶ to R¹⁰ may also form, together with the carbon atoms that bear them, a saturated 5- or 6-membered (hetero)cycle.

More preferentially, according to this third variant, R⁶ to R¹⁰ represent, independently of each other:
a hydrogen atom;
a (C₁-C₄)alkyl group;
a (C₁-C₄)alkoxy group;
a hydroxy(C₁-C₄)alkyl group;
a hydroxyl group.

Preferably, according to this third variant, R^{b} represents:
a hydrogen atom;
a (C₁-C₄)alkyl group;
a hydroxy(C₁-C₄)alkyl group;
a (C₁-C₄)alkoxy group;
a halogen atom;
a hydroxyl group;
an —O—C(O)—CH₃ group;

it being understood that:
R^{b} may form with R⁶ or R¹⁰ and the carbon atoms that bear them a 4- to 8-membered (hetero)cycle, more particularly a 5- to 6-membered carbocycle.

More preferentially, according to this third variant, R^{b} represents:
a hydrogen atom;
a (C₁-C₄)alkyl group;
a (C₁-C₄)alkoxy group;
a hydroxy(C₁-C₄)alkyl group;
a hydroxyl group.

### Fourth Variant

According to a fourth variant, the compounds of formula (IIb), (IIIb) or (IVb) are respectively chosen from the compounds of formula (IIb₄), (IIIb₄) or (IVb₄) below:

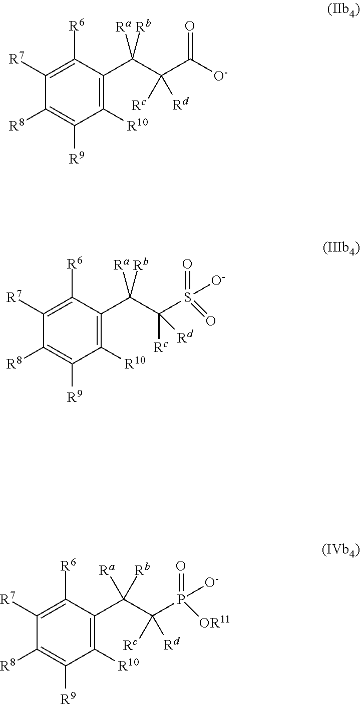

in which:
R⁶ to R¹⁰ represent, independently of each other:
   a hydrogen atom;
   a (C₁-C₆)alkyl or (C₁-C₆)alkoxy group, the (C₁-C₆)alkyl or (C₁-C₆)alkoxy group being optionally substituted with one or more identical or different groups chosen from: —OR, —NR′R″, —C(O)OR, —O—C(O)—R, aryl and —CF₃ and/or optionally interrupted with one or more groups or heteroatoms chosen from: O, NR′, CO, S, SO and SO₂; R representing a hydrogen atom or a (C₁-C₄)alkyl group, R′ and R″ representing, independently of each other, a hydrogen atom, a (C₁-C₄)alkyl group or a (C₁-C₄)alkylcarbonyl group such as acetyl, it being understood that the group —NR′R″ is other than an —NH₂ group;
   a halogen atom;
   a group —O—X, in which X represents a hydrogen atom or a protecting group for the phenol function such as an acyl group, in particular an acetyl group;

it being understood that two adjacent groups R⁶ to R¹⁰ may also form, together with the carbon atoms that bear them, a saturated or unsaturated or aromatic 5- or 6-membered (hetero)cycle;
R^{a}, R^{b}, R^{c} and R^{d} represent, independently of each other:
   a hydrogen atom;
   a (C₁-C₆)alkyl group optionally substituted with one or more identical or different groups chosen from: —OR, —NR′R″, —C(O)OR, —O—C(O)—R, (hetero)cycle, —C(O)—R, —NH—C(O)—R, —C(O)—NH—R, with R, R′ and R″ as defined previously, it being understood that the group —NR′R″ is other than an —NH₂ group;
   a (C₁-C₆)alkoxy group optionally substituted with one or more identical or different groups chosen from: —OR, —NR′R″, —C(O)OR, —O—C(O)—R, aryl and —CF₃ and/or optionally interrupted with one or more groups or heteroatoms chosen from: O, NR′, CO, S, SO and SO₂, with R, R′ and R″ as defined previously, it being understood that the group —NR′R″ is other than an —NH₂ group;
   a halogen atom;
   a group —O—X with X as defined previously;

it being understood that:
(R^{a} and R^{b}) and/or (R^{c} and R^{d}) may form, together with the carbon atom which bears them, a C═O bond;
R^{a} and R^{b} may form, together with the carbon atom which bears them, a saturated or unsaturated, 4- to 8-membered, in particular 4- to 6-membered, (hetero)cycle, and more particularly a 5- or 6-membered carbocycle;
R^{a} or R^{b} or R^{c} or R^{d} may form with R⁶ or R¹⁰ and the carbon atoms that bear them a 4- to 8-membered (hetero)cycle, more particularly a 5- to 6-membered carbocycle;
R¹¹ represents a hydrogen atom, a negative charge, an alkali metal or an alkaline-earth metal, preferably a negative charge.

Preferably, according to this fourth variant, R⁶ to R¹⁰ represent, independently of each other:
a hydrogen atom;
a (C₁-C₄)alkyl group;
a (C₁-C₄)alkoxy group;
a hydroxy(C₁-C₄)alkyl group;
a halogen atom;
a hydroxyl group;
an —O—C(O)—CH₃ group;

it being understood that two adjacent groups R⁶ to R¹⁰ may also form, together with the carbon atoms that bear them, a saturated 5- or 6-membered (hetero)cycle.

More preferentially, according to this fourth variant, R⁶ to R¹⁰ represent, independently of each other:
a hydrogen atom;
a (C₁-C₄)alkyl group;
a (C₁-C₄)alkoxy group;
a hydroxy(C₁-C₄)alkyl group;
a hydroxyl group.

Preferably, according to this fourth variant, R^{a}, R^{b}, R^{c} and R^{d} represent, independently of each other:
a hydrogen atom;
a (C₁-C₄)alkyl group;
a hydroxy(C₁-C₄)alkyl group;
a (C₁-C₄)alkoxy group;
a halogen atom;
a hydroxyl group;
an —O—C(O)—CH₃ group;

it being understood that:
(R^{a} and R^{b}) and/or (R^{c} and R^{d}) may form, together with the carbon atom which bears them, a C═O bond;
R^{a} or R^{b} or R^{c} or R^{d} may form with R⁶ or R¹⁰ and the carbon atoms that bear them a 4- to 8-membered (hetero)cycle, more particularly a 5- to 6-membered carbocycle.

More preferentially, according to this fourth variant, R^{a}, R^{b}, R^{c} and R^{d} represent, independently of each other:
a hydrogen atom;
a (C₁-C₄)alkyl group;
a (C₁-C₄)alkoxy group;
a hydroxy(C₁-C₄)alkyl group;
a hydroxyl group.

### Fifth Variant

According to a fifth variant, the compounds of formula (IIb), (IIIb) or (IVb) are respectively chosen from the compounds of formula (IIb₅), (IIIb₅) or (IVb₅) below:

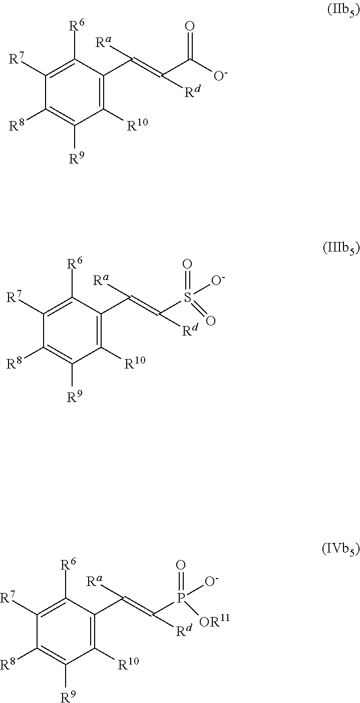

in which:
R⁶ to R¹⁰ represent, independently of each other:
   a hydrogen atom;
   a (C₁-C₆)alkyl or (C₁-C₆)alkoxy group, the (C₁-C₆)alkyl or (C₁-C₆)alkoxy group being optionally substituted with one or more identical or different groups chosen from: —OR, —NR′R″, —C(O)OR, —O—C(O)—R, aryl and —CF₃ and/or optionally interrupted with one or more groups or heteroatoms chosen from: O, NR′, CO, S, SO and SO₂; R representing a hydrogen atom or a (C₁-C₄)alkyl group, R′ and R″ representing, independently of each other, a hydrogen atom, a (C₁-C₄)alkyl group or a (C₁-C₄)alkylcarbonyl group such as acetyl, it being understood that the group —NR′R″ is other than an —NH₂ group;
   a halogen atom;
   a group —O—X, in which X represents a hydrogen atom or a protecting group for the phenol function such as an acyl group, in particular an acetyl group;

it being understood that two adjacent groups R⁶ to R¹⁰ may also form, together with the carbon atoms that bear them, a saturated or unsaturated or aromatic 5- or 6-membered (hetero)cycle:
R^{a} and R^{d} represent, independently of each other:
   a hydrogen atom;
   a (C₁-C₆)alkyl group optionally substituted with one or more identical or different groups chosen from: —OR, —NR′R″, —C(O)OR, —O—C(O)—R, (hetero)cycle, —C(O)—R, —NH—C(O)—R, —C(O)—NH—R, with R, R′ and R″ as defined previously, it being understood that the group —NR′R″ is other than an —NH₂ group;
   a (C₁-C₆)alkoxy group optionally substituted with one or more identical or different groups chosen from: —OR, —NR′R″, —C(O)OR, —O—C(O)—R, aryl and —CF₃ and/or optionally interrupted with one or more groups or heteroatoms chosen from: O, NR′, CO, S, SO and SO₂, with R, R′ and R″ as defined previously, it being understood that the group —NR′R″ is other than an —NH₂ group;
   a halogen atom;
   a group —O—X with X as defined previously;

it being understood that:
R¹¹ represents a hydrogen atom, a negative charge, an alkali metal or an alkaline-earth metal, preferably a negative charge.

The ethylenic double bond between —CR^{a} and —CR^{c} may be cis or trans.

Preferably, according to this fifth variant, R⁶ to R¹⁰ represent, independently of each other:
a hydrogen atom;
a (C₁-C₄)alkyl group;
a (C₁-C₄)alkoxy group;
a hydroxy(C₁-C₄)alkyl group;
a halogen atom;
a hydroxyl group;
an —O—C(O)—CH₃ group;

it being understood that two adjacent groups R⁶ to R¹⁰ may also form, together with the carbon atoms that bear them, a saturated 5- or 6-membered (hetero)cycle.

More preferentially, according to this fifth variant, R⁶ to R¹⁰ represent, independently of each other:
a hydrogen atom;
a (C₁-C₄)alkyl group;
a (C₁-C₄)alkoxy group;
a hydroxy(C₁-C₄)alkyl group;
a hydroxyl group.

Preferably, according to this fifth variant, R^{a} and R^{c} represent, independently of each other:
a hydrogen atom;
a (C₁-C₄)alkyl group;
a hydroxy(C₁-C₄)alkyl group;
a (C₁-C₄)alkoxy group;
a halogen atom;
a hydroxyl group;
an —O—C(O)—CH₃ group.

More preferentially, according to this fifth variant, R^{a} and R^{c} represent, independently of each other:
a hydrogen atom;
a (C₁-C₄)alkyl group;
a (C₁-C₄)alkoxy group;
a hydroxy(C₁-C₄)alkyl group;
a hydroxyl group.

### Sixth Variant

According to a sixth variant, the compounds of formula (IIb), (IIIb) or (IVb) are respectively chosen from the compounds of formula (IIb₆), (IIIb₆) or (IVb₆) below:

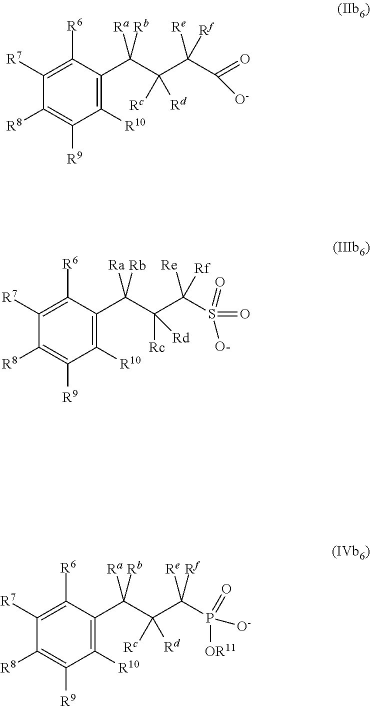

in which:
R⁶ to R¹⁰ represent, independently of each other:
   a hydrogen atom;
   a (C₁-C₆)alkyl or (C₁-C₆)alkoxy group, the (C₁-C₆)alkyl or (C₁-C₆)alkoxy group being optionally substituted with one or more identical or different groups chosen from: —OR, —NR′R″, —C(O)OR, —O—C(O)—R, aryl and —CF₃ and/or optionally interrupted with one or more groups or heteroatoms chosen from: O, NR′, CO, S, SO and SO₂; R representing a hydrogen atom or a (C₁-C₄)alkyl group, R′ and R″ representing, independently of each other, a hydrogen atom, a (C₁-C₄)alkyl group or a (C₁-C₄)alkylcarbonyl group such as acetyl, it being understood that the group —NR′R″ is other than an —NH₂ group;
   a halogen atom;
   a group —O—X, in which X represents a hydrogen atom or a protecting group for the phenol function such as an acyl group, in particular an acetyl group;

it being understood that two adjacent groups R⁶ to R¹⁰ may also form, together with the carbon atoms that bear them, a saturated or unsaturated or aromatic 5- or 6-membered (hetero)cycle;
R^{a}, R^{b}, R^{c}, R^{d}, R^{e} and R^{f} represent, independently of each other:
   a hydrogen atom;
   a (C₁-C₆)alkyl group optionally substituted with one or more identical or different groups chosen from: —OR, —NR′R″, —C(O)OR, —O—C(O)—R, (hetero)cycle, —C(O)—R, —NH—C(O)—R, —C(O)—NH—R, with R, R′ and R″ as defined previously, it being understood that the group —NR′R″ is other than an —NH₂ group;
   a (C₁-C₆)alkoxy group optionally substituted with one or more identical or different groups chosen from: —OR, —NR′R″, —C(O)OR, —O—C(O)—R, aryl and —CF₃ and/or optionally interrupted with one or more groups or heteroatoms chosen from: O, NR′, CO, S, SO and SO₂, with R, R′ and R″ as defined previously, it being understood that the group —NR′R″ is other than an —NH₂ group;
   a halogen atom;
   a group —O—X with X as defined previously;

it being understood that:
(R^{a} and R^{b}) and/or (R^{c} and R^{d}) may form, together with the carbon atom which bears them, a C═O bond;
R^{a} and R^{b} may form, together with the carbon atom which bears them, a saturated or unsaturated, 4- to 8-membered, in particular 4- to 6-membered, (hetero)cycle, and more particularly a 5- or 6-membered carbocycle;
R^{a} or R^{b} or R^{c} or R^{d} may form with R⁶ or R¹⁰ and the carbon atoms that bear them a 4- to 8-membered (hetero)cycle, more particularly a 5- to 6-membered carbocycle;
R¹¹ represents a hydrogen atom, a negative charge, an alkali metal or an alkaline-earth metal, preferably a negative charge.

Preferably, according to this sixth variant, R⁶ to R¹⁰ represent, independently of each other:
a hydrogen atom;
a (C₁-C₄)alkyl group;
a (C₁-C₄)alkoxy group;
a hydroxy(C₁-C₄)alkyl group;
a halogen atom;
a hydroxyl group;
an —O—C(O)—CH₃ group;

it being understood that two adjacent groups R⁶ to R¹⁰ may also form, together with the carbon atoms that bear them, a saturated 5- or 6-membered (hetero)cycle.

More preferentially, according to this sixth variant, R⁶ to R¹⁰ represent, independently of each other:
a hydrogen atom;
a (C₁-C₄)alkyl group;
a (C₁-C₄)alkoxy group;
a hydroxy(C₁-C₄)alkyl group;
a hydroxyl group.

Preferably, according to this sixth variant, R^{a}, R^{b}, R^{c}, R^{d}, R^{e} and R^{f} represent, independently of each other:
a hydrogen atom;
a (C₁-C₄)alkyl group;
a hydroxy(C₁-C₄)alkyl group;
a (C₁-C₄)alkoxy group;
a halogen atom;
a hydroxyl group;
an —O—C(O)—CH₃ group;

it being understood that:
(R^{a} and R^{b}) and/or (R^{c} and R^{d}) may form, together with the carbon atom which bears them, a C═O bond;
R^{a} or R^{b} or R^{c} or R^{d} may form with R⁶ or R¹⁰ and the carbon atoms that bear them a 4- to 8-membered (hetero)cycle, more particularly a 5- to 6-membered carbocycle.

More preferentially, according to this sixth variant, R^{a}, R^{b}, R^{c}, R^{d}, R^{e} and R^{f} represent, independently of each other:
a hydrogen atom;
a (C₁-C₄)alkyl group;
a (C₁-C₄)alkoxy group;
a hydroxy(C₁-C₄)alkyl group;
a hydroxyl group.

### Compounds of Formula (IIc)

In the compounds of formula (IIc), the substituents R⁷ may be identical or different.

Preferably, R⁶ and the substituents R⁷ represent, independently of each other, a hydrogen atom or a group chosen from:
a group of formula (a) or (b) or (c) as defined above, preferably a group of formula (a);
a hydroxyl group;
a (C₁-C₄)alkoxy group.

### First Variant

According to a first variant, the compounds of formula (IIc) are such that R⁶ and at least one of the substituents R⁷ represent a group of formula (a) or (b) or (c), preferably a group of formula (a).

### Second Variant

According to a second variant, the compounds of formula (IIc) are such that each of the X¹, X², X³ and X⁴ represents CR⁷ with R⁷ as defined above.

According to a particular embodiment of this second variant, R⁶ represents a group of formula (a) or (b) or (c), preferably a group of formula (a).

### Third Variant

According to a third variant, the compounds of formula (IIc) are such that only one from among X¹, X², X³ and X⁴ represents a nitrogen atom.

According to a particular embodiment of this third variant, R⁶ represents a group of formula (a) or (b) or (c), preferably a group of formula (a).

According to another particular embodiment of this third variant, R⁶ represents a group of formula (a) or (b) or (c), preferably a group of formula (a) and R⁷ independently represents a hydrogen atom, a hydroxyl group or a group of formula (a).

According to a particular embodiment, the compounds of formula (IIc) are such that only one from among X¹, X² and X³ represents a nitrogen atom.

### Fourth Variant

According to a fourth variant, the compounds of formula (IIc) are such that two and only two from among X¹, X², X³ and X⁴ represent a nitrogen atom.

According to a particular embodiment of this fourth variant, the compounds of formula (IIc) are such that X² represents a nitrogen atom and X³ or X⁴ represents a nitrogen atom.

According to a particular embodiment of this fourth variant, R⁶ represents a group of formula (a) or (b) or (c), preferably a group of formula (a).

Composition (A) and/or (B) and/or (C) may comprise a mixture of compounds D comprising different anions An⁻ such as those described above.

Preferably, An⁻ represents an organic anion chosen from the compounds of formulae 1 to 46 below:

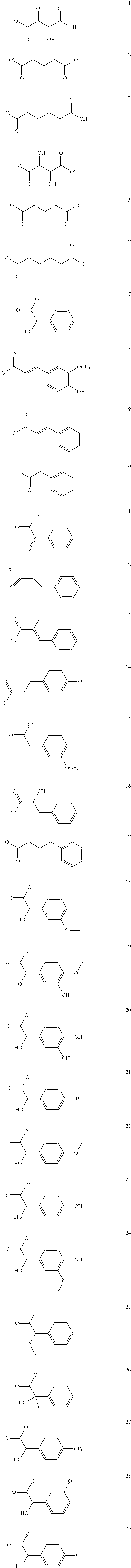

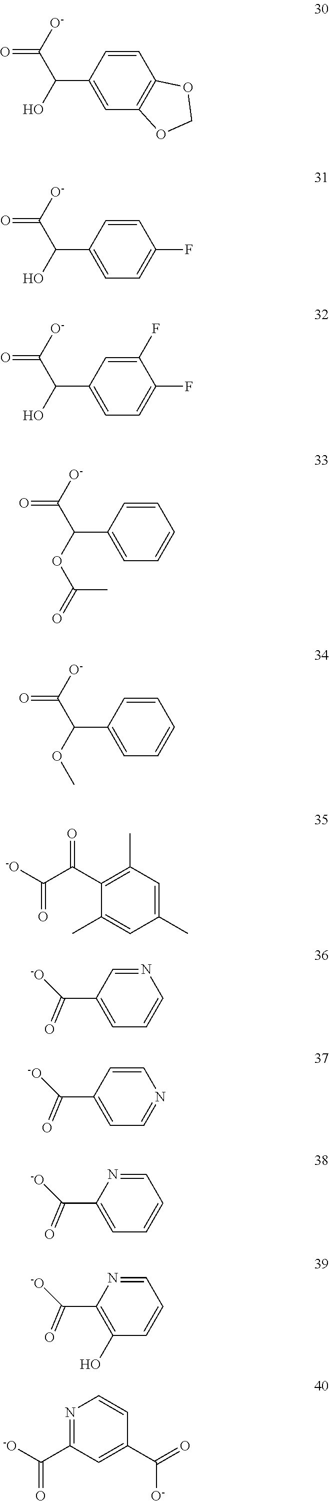

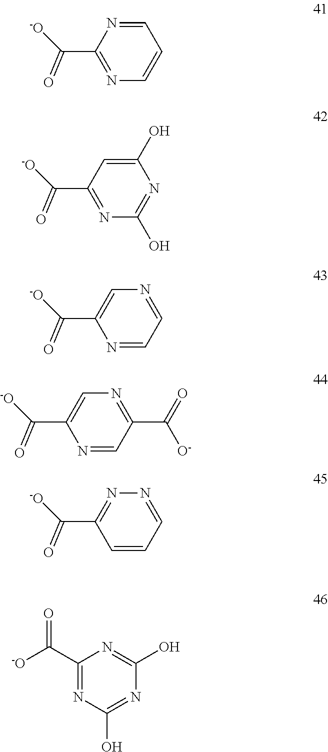

more preferentially chosen from the compounds of formula 4, 7, 10 or 36, even more preferentially chosen from the compounds of formula 4, 7 or 10.

According to a preferred embodiment, An⁻ represents an organic anion chosen from the compounds of formulae 1 to 6 below:

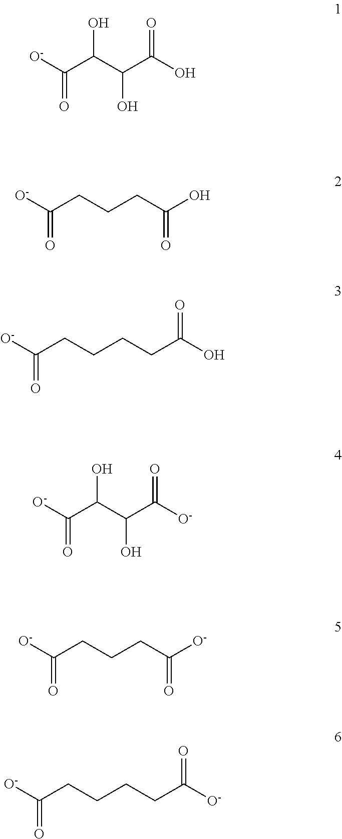 [SMILES: O=C([O-])C(O)C(O)C(=O)O ; O=C([O-])CCCC(=O)O ; O=C([O-])CCCCC(=O)O ; O=C([O-])C(O)C(O)C(=O)[O-] ; O=C([O-])CCCC(=O)[O-] ; O=C([O-])CCCCC(=O)[O-]]

According to this preferred embodiment, An⁻ preferably represents an organic anion of formula 4.

According to another preferred embodiment, An⁻ represents an organic anion chosen from the compounds of formulae 7 to 35 below:

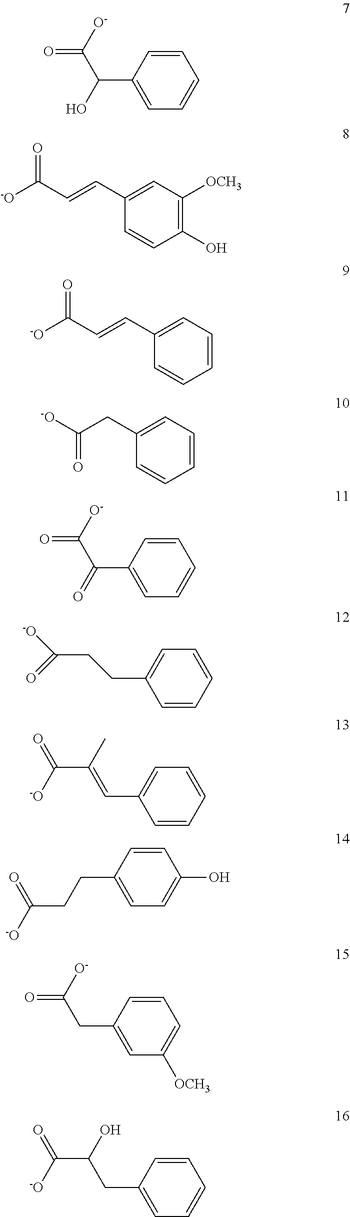

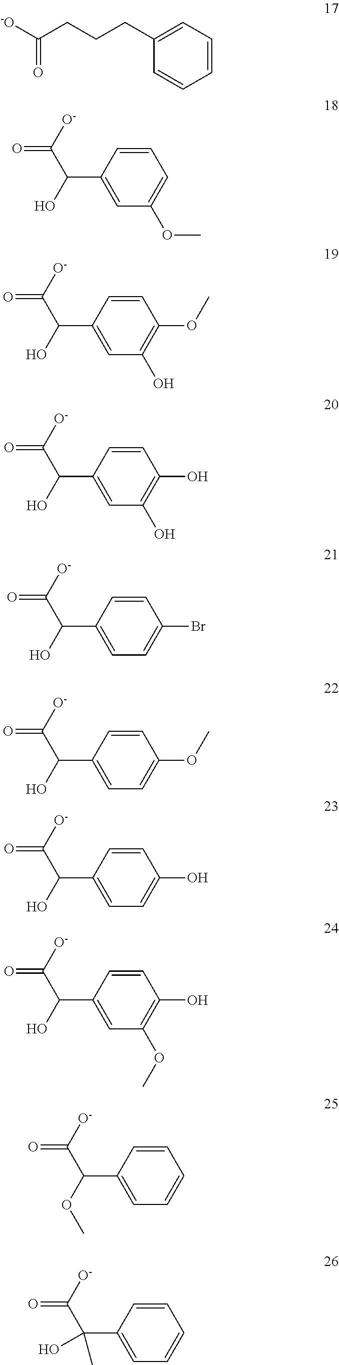

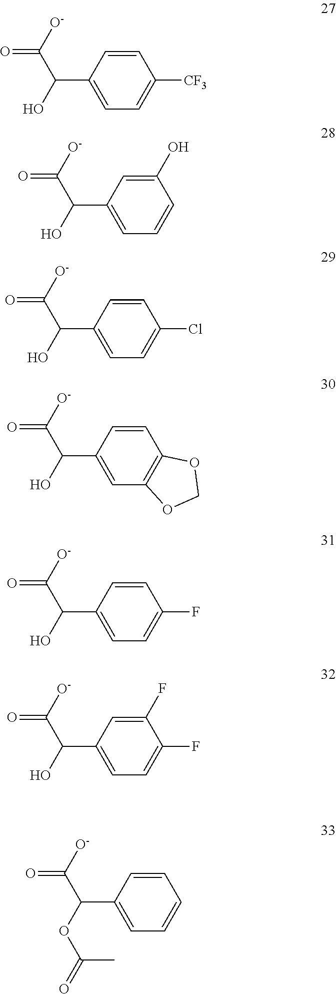

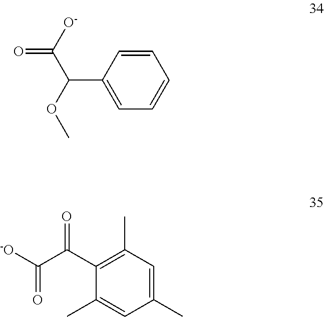

According to this other preferred embodiment, An⁻ preferably represents an organic anion of formula 7 or 10.

According to another preferred embodiment, An⁻ represents an organic anion chosen from the compounds of formulae 36 to 46 below:

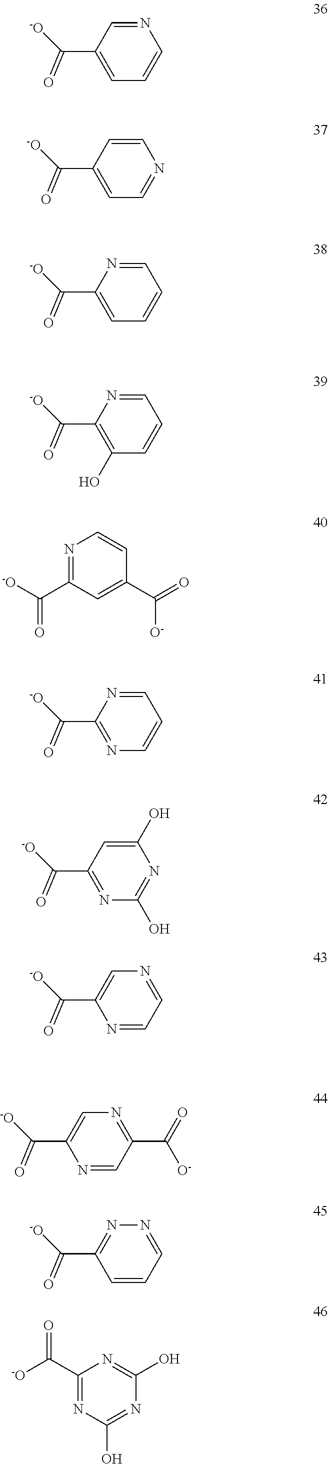

According to this other preferred embodiment, An⁻ preferably represents an organic anion of formula 36.

### Cationic Portion

In the case where R⁵ represents an alkali metal, it may be sodium.

Preferably, the compounds of formula (I) are carnitine salts. Thus, preferably, R¹, R² and R³ represent a methyl group and R⁴ and R⁵ represent a hydrogen atom.

Preferably, the cationic portion of the compound of formula (I) is in the form of an optical isomer of L (laevorotatory) or D (dextrorotatory) configuration, more preferentially of L configuration.

A compound of formula (I) may be obtained by extemporaneously mixing a first composition comprising the cationic portion of the compound of formula (I) optionally in the form of a salt and a second composition comprising the anionic portion of the compound of formula (I) optionally in the form of a salt. The mixing may be extemporaneous.

The compounds of formula (I) are preferably chosen from the compounds of formulae 1′ to 46′ below:

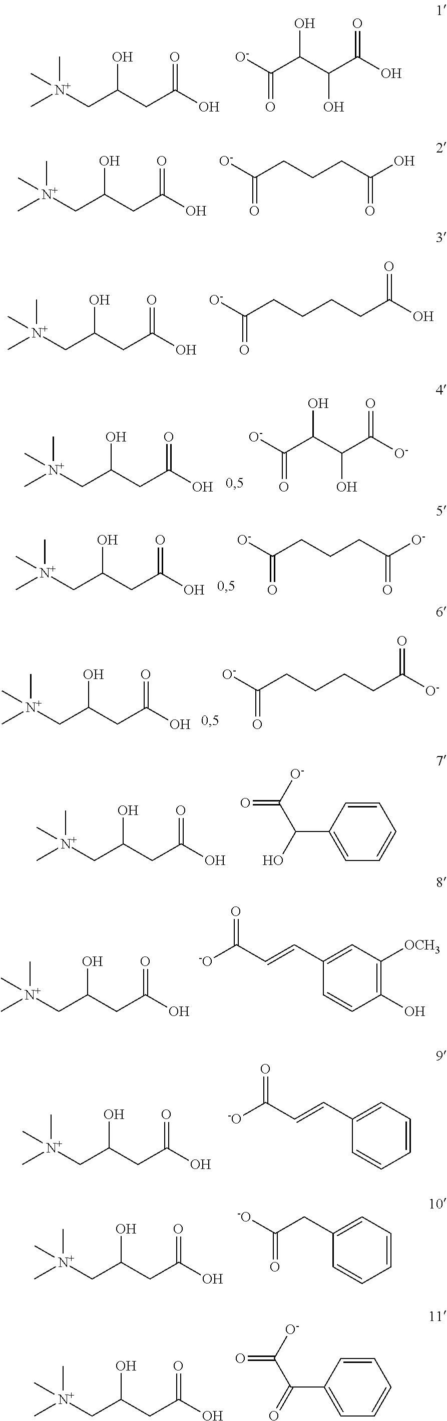

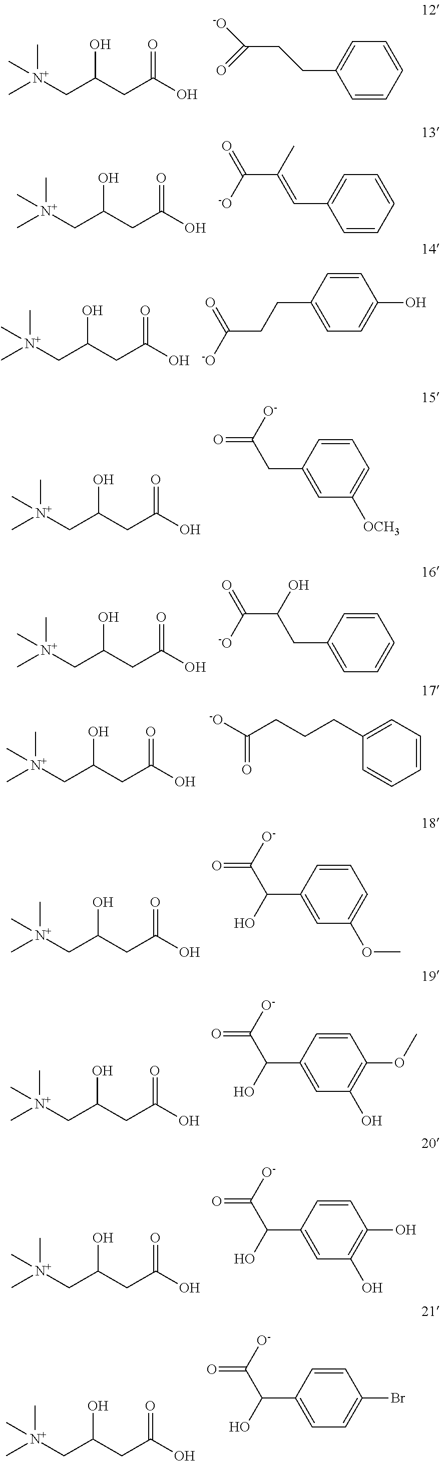

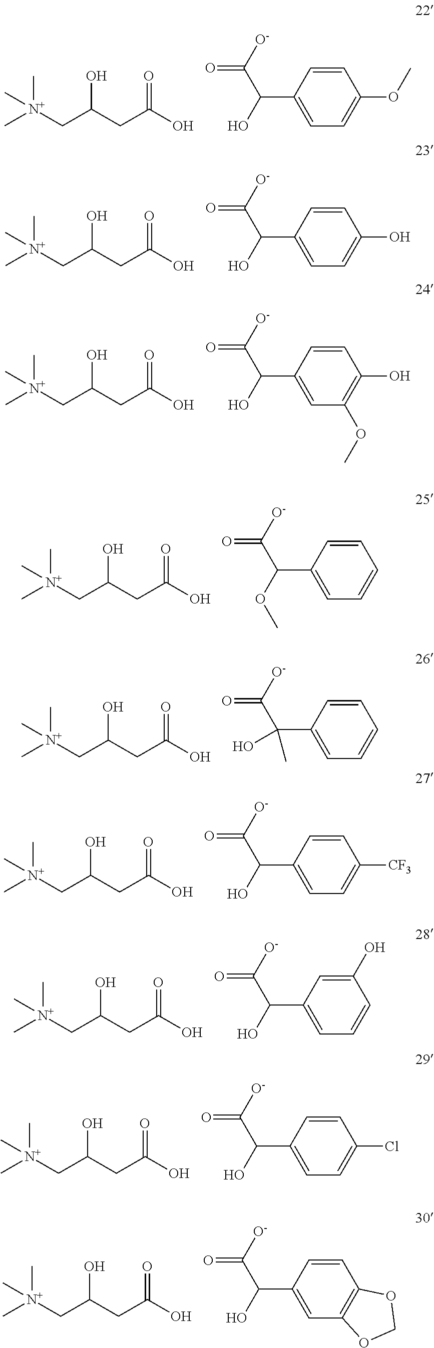

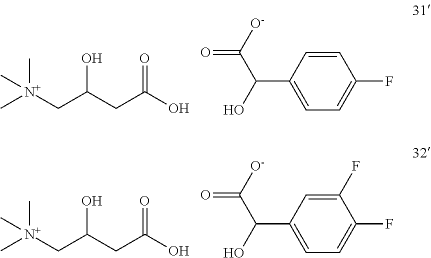

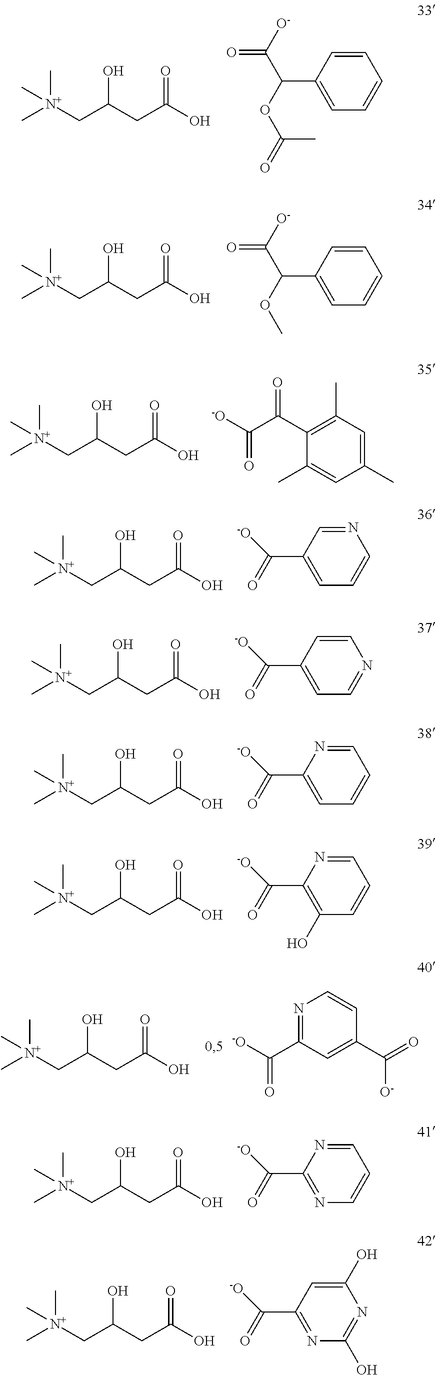

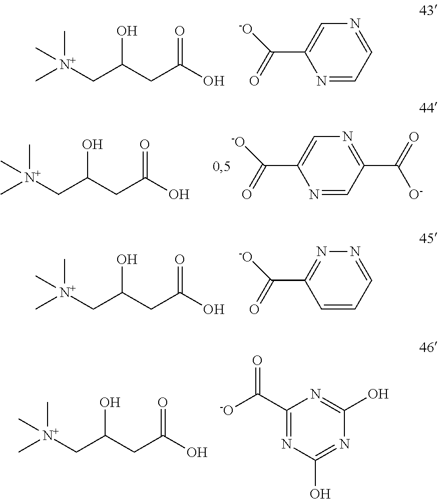

more preferentially chosen from the compounds of formula 4′, 7′, 10′ or 36′, even more preferentially chosen from the compounds of formula 4′, 7′ or 10′.

According to a preferred embodiment, composition (A) comprises a total content of colouring agents of less than 0.1% by weight, preferably less than 0.01% by weight, more preferentially less than 0.001% by weight relative to the total weight of composition (A), and/or composition (C) comprises a total content of colouring agents of less than 0.1% by weight, preferably less than 0.01% by weight, more preferentially less than 0.001% by weight relative to the total weight of composition (C).

According to a more preferred embodiment, composition (A) and/or composition (C) is free of colouring agents.

### pH of Compositions (A) and (C)

The pH of composition (A) is preferably greater than 7, more preferentially ranging from 7 to 10, even more preferentially ranging from 7.5 to 9.5.

The pH of composition (C) is preferably less than 7, more preferentially ranging from 3 to 7, even more preferentially ranging from 3.1 to 6.5, most preferentially ranging from 3.5 to 6.

### Composition (B)

According to a preferred embodiment, composition (B) comprises:
i) hydrogen peroxide;
ii) one or more compounds chosen from carbonates, bicarbonates and mixtures thereof;
iii) one or more silicates.

### Chemical Oxidizing Agents

Composition (B) comprises i) one or more chemical oxidizing agents chosen from hydrogen peroxide, hydrogen peroxide-generating systems other than peroxygenated salts, and mixtures thereof.

The hydrogen peroxide-generating systems other than peroxygenated salts may be chosen from urea peroxide, polymeric complexes that can release hydrogen peroxide, oxidases, and mixtures thereof.

As examples of polymeric complexes that can release hydrogen peroxide, mention may be made of polyvinylpyrrolidone/H₂O₂ in particular in powder form, and the other polymeric complexes described in U.S. Pat. Nos. 5,008,093, 3,376,110 and 5,183,901.

Oxidases can produce hydrogen peroxide in the presence of a suitable substrate, for instance glucose in the case of glucose oxidase or uric acid with uricase.

According to a particular embodiment, hydrogen peroxide and/or the hydrogen peroxide-generating system(s) other than peroxygenated salts may be incorporated in the composition according to the invention just before it is applied to the keratin fibres. The intermediate composition(s) comprising the hydrogen peroxide and/or the hydrogen peroxide-generating system(s) other than peroxygenated salts can be referred to as oxidizing compositions and can also contain various additional compounds or various adjuvants conventionally used in compositions for lightening keratin fibres.

According to a preferred embodiment, composition (B) comprises hydrogen peroxide as chemical oxidizing agent.

The chemical oxidizing agent(s) are present in composition (B) preferably in a total content ranging from 1% to 12% by weight, more preferentially ranging from 3% to 9% by weight, even more preferentially ranging from 3.5% to 8.5% by weight, relative to the total weight of composition (B).

According to a preferred embodiment, the hydrogen peroxide is present in composition (B) in a total content ranging from 1% to 12% by weight, preferably ranging from 3% to 9% by weight, more preferentially ranging from 3.5% to 8.5% by weight, relative to the total weight of composition (B).

(Bi)Carbonates and/or (Bi)Carbonate-Generating Systems

Composition (B) further comprises ii) one or more compounds chosen from carbonates, carbonate-generating systems, bicarbonates, bicarbonate-generating systems and mixtures thereof.

According to a preferred embodiment, composition (B) further comprises ii) one or more compounds chosen from carbonates, bicarbonates and mixtures thereof.

According to a more preferred embodiment, composition (B) further comprises ii) one or more compounds chosen from ammonium carbonate, ammonium bicarbonate and mixtures thereof.

The compound(s) ii) are present in composition (B) preferably in a total content ranging from 0.01% to 20% by weight, preferably ranging from 1% to 15% by weight, more preferentially ranging from 2% to 15% by weight, even more preferentially ranging from 4% to 15% by weight, relative to the total weight of composition (B).

Carbonates and/or Carbonate-Generating Systems

The term “carbonate-generating system” means a system which generates carbonate in situ, for instance carbon dioxide in water or percarbonate in water.

Preferably, the carbonate(s) are chosen from:
alkali metal carbonates;
alkaline-earth metal carbonates;
lanthanide carbonates;
transition metal carbonates;
bismuth carbonate;
cadmium carbonate;
thallium carbonate;
zinc carbonate;
the compounds of formula (N⁺R¹R²R³R⁴)₂, ·CO₃²⁻ in which R¹, R², R³ and R⁴ represent, independently of one another, a hydrogen atom or a (C₁-C₄)alkyl group optionally substituted by a hydroxyl group;
guanidine carbonate;
mixtures thereof.

More preferentially, the carbonate(s) are chosen from sodium carbonate, potassium carbonate, caesium carbonate, lithium carbonate, magnesium carbonate, calcium carbonate, barium carbonate, strontium carbonate, cerium carbonate, lanthanum carbonate, yttrium carbonate, copper(II) carbonate, manganese carbonate, nickel carbonate, silver carbonate, zirconium carbonate, bismuth carbonate, cadmium carbonate, thallium carbonate, zinc carbonate, ammonium carbonate, guanidine carbonate, tetraethylammonium carbonate, and mixtures thereof.

Even more preferentially, the carbonate(s) are chosen from sodium carbonate, potassium carbonate, caesium carbonate, magnesium carbonate, calcium carbonate, cerium carbonate, manganese carbonate, zinc carbonate, ammonium carbonate, guanidine carbonate, and mixtures thereof.

Most preferentially, the carbonate(s) are chosen from sodium carbonate, potassium carbonate, magnesium carbonate, calcium carbonate, ammonium carbonate, and mixtures thereof.

According to a particularly preferred embodiment, the carbonate included in composition (B) is ammonium carbonate.

The carbonate(s) and/or the carbonate-generating system(s) are present in composition (B) preferably in a total content ranging from 0.01% to 20% by weight, more preferentially ranging from 1% to 20% by weight, even more preferentially ranging from 1% to 10% by weight, relative to the total weight of composition (B).

According to a preferred embodiment, the carbonate(s) are present in composition (B) in a total content ranging from 0.01% to 20% by weight, preferably ranging from 1% to 20% by weight, more preferentially ranging from 1% to 10% by weight, relative to the total weight of composition (B).

According to a preferred embodiment, the compound(s) ii) are chosen from carbonates, carbonate-generating systems and mixtures thereof, preferably from carbonates.

Bicarbonates and/or Bicarbonate-Generating Systems

The term “bicarbonate-generating system” means a system which generates bicarbonate in situ, for instance carbon dioxide in water or by buffering a carbonate with a mineral or organic acid.

Preferably, the bicarbonate(s) are chosen from:
alkali metal bicarbonates;
alkaline-earth metal bicarbonates;
the compounds of formula N⁺R¹R²R³R⁴, ·HCO₃⁻ in which R¹, R², R³ and R⁴ represent, independently of one another, a hydrogen atom or a (C₁-C₄)alkyl group optionally substituted by a hydroxyl group;
aminoguanidine bicarbonate;
mixtures thereof.

More preferentially, the bicarbonate(s) are chosen from sodium bicarbonate, potassium bicarbonate, lithium bicarbonate, caesium bicarbonate, calcium bicarbonate, magnesium bicarbonate, ammonium bicarbonate, choline bicarbonate, triethylammonium bicarbonate, aminoguanidine bicarbonate, and mixtures thereof.

Even more preferentially, the bicarbonate(s) are chosen from sodium bicarbonate, potassium bicarbonate, caesium bicarbonate, calcium bicarbonate, magnesium bicarbonate, ammonium bicarbonate, and mixtures thereof.

Most preferentially, the bicarbonate(s) are chosen from sodium bicarbonate, potassium bicarbonate, ammonium bicarbonate, and mixtures thereof.

According to a particularly preferred embodiment, the bicarbonate included in composition (B) is ammonium bicarbonate.

The bicarbonates may originate from a natural water, for example spring water from the Vichy basin or from La Roche Posay or Badoit water.

The bicarbonate(s) and/or the bicarbonate-generating system(s) are present in composition (B) preferably in a total content ranging from 0.01% to 20% by weight, more preferentially ranging from 1% to 15% by weight, even more preferentially ranging from 2% to 15% by weight, most preferentially ranging from 4% to 15% by weight, relative to the total weight of composition (B).

According to a preferred embodiment, the bicarbonate(s) are present in a total content ranging from 0.01% to 20% by weight, preferably ranging from 1% to 15% by weight, more preferentially ranging from 2% to 15% by weight, even more preferentially ranging from 4% to 15% by weight, relative to the total weight of composition (B).

According to a preferred embodiment, the compound(s) ii) are chosen from bicarbonates, bicarbonate-generating systems and mixtures thereof, preferably from bicarbonates.

### Silicates

Composition (B) further comprises iii) one or more silicates.

The silicate(s) are preferably water-soluble.

The term “water-soluble silicate” means a silicate which has a solubility in water at ordinary room temperature (25° C.) and at atmospheric pressure (760 mmHg) of greater than 0.5% by weight, preferably greater than 1% by weight.

Preferably, the silicate(s) are chosen from alkali metal silicates, alkaline-earth metal silicates, aluminium silicates, trimethylammonium silicates, and mixtures thereof.

More preferentially, the silicate(s) are chosen from sodium silicates, potassium silicates, calcium silicates, aluminium silicates, trimethylammonium silicates, and mixtures thereof.

Even more preferentially, the silicate(s) are chosen from sodium silicates.

Preferably, the silicate(s) are chosen from compounds having the INCI name Sodium Silicate (CAS: [1344-09-8]) and/or Sodium Metasilicate (CAS: [6834-92-0]).

The silicate(s) are present in composition (B) preferably in a total content ranging from 1% to 40% by weight, more preferentially ranging from 2% to 35% by weight, even more preferentially ranging from 3% to 35% by weight, most preferentially ranging from 4% to 20% by weight, relative to the total weight of composition (B).

The weight ratio of total amount of carbonate(s) and/or carbonate-generating system(s)/total amount of silicate(s) in composition (B) is preferably from 0.00025 to 20, more preferentially from 0.028 to 10, even more preferentially from 0.028 to 3.4.

According to a preferred embodiment, the weight ratio of total amount of carbonate(s)/total amount of silicate(s) in composition (B) is from 0.00025 to 20, preferably from 0.028 to 10, more preferentially from 0.028 to 3.4.

The weight ratio of total amount of carbonate(s) and/or carbonate-generating system(s)/total amount of chemical oxidizing agent(s) in composition (B) is preferably from 0.0008 to 20, more preferentially from 0.1 to 6.6, even more preferentially from 0.1 to 2.9.

According to a preferred embodiment, the weight ratio of total amount of carbonate(s)/total amount of chemical oxidizing agent(s) in composition (B) is from 0.0008 to 20, preferably from 0.1 to 6.6, more preferentially from 0.1 to 2.9.

According to a preferred embodiment, the weight ratio of total amount of carbonate(s)/total amount of hydrogen peroxide in composition (B) is from 0.0008 to 20, preferably from 0.1 to 6.6, more preferentially from 0.1 to 2.9.

The weight ratio of total amount of bicarbonate(s) and/or bicarbonate-generating system(s)/total amount of silicate(s) in composition (B) is preferably from 0.00025 to 20, more preferentially from 0.02 to 7.5, even more preferentially from 0.05 to 5.

According to a preferred embodiment, the weight ratio of total amount of bicarbonate(s)/total amount of silicate(s) in composition (B) is from 0.00025 to 20, preferably from 0.02 to 7.5, more preferentially from 0.05 to 5.

The weight ratio of total amount of bicarbonate(s) and/or bicarbonate-generating system(s)/total amount of chemical oxidizing agent(s) in composition (B) is preferably from 0.0008 to 20, more preferentially from 0.11 to 5, even more preferentially from 0.2 to 4.2.

According to a preferred embodiment, the weight ratio of total amount of bicarbonate(s)/total amount of chemical oxidizing agent(s) in composition (B) is from 0.0008 to 20, preferably from 0.11 to 5, more preferentially from 0.2 to 4.2.

According to a more preferred embodiment, the weight ratio of total amount of bicarbonate(s)/total amount of hydrogen peroxide in composition (B) is from 0.0008 to 20, preferably from 0.11 to 5, more preferentially from 0.2 to 4.2.

The weight ratio of total amount of carbonate(s) and/or carbonate-generating system(s)/total amount of bicarbonate(s) and/or bicarbonate-generating system(s) in composition (B) is preferably from 0.0005 to 2000, more preferentially from 0.06 to 20, even more preferentially from 0.06 to 5.

According to a preferred embodiment, the weight ratio of total amount of carbonate(s)/total amount of bicarbonate(s) in composition (B) is from 0.0005 to 2000, preferably from 0.06 to 20, more preferentially from 0.06 to 5.

Composition (B) preferably comprises a total content of magnesium carbonate of less than 5% by weight, more preferentially of less than 1% by weight, even more preferentially of less than 0.1% by weight, most preferentially of less than 0.01% by weight, and better still of less than 0.001% by weight.

According to a particularly preferred embodiment, the composition is free of magnesium carbonate.

Composition (B) preferably comprises a total content of persulfates of less than 10% by weight, more preferentially of less than 5% by weight, even more preferentially of less than 1% by weight, most preferentially of less than 0.1% by weight, and better still of less than 0.01% by weight, and even better still of less than 0.001% by weight.

According to a particularly preferred embodiment, the composition is free of persulfates.

According to a particular embodiment, composition (B) further comprises iv) one or more colouring agents, preferably chosen from direct dyes, oxidation dyes and mixtures thereof.

According to this particular embodiment, the process according to the invention is a process for simultaneous bleaching and dyeing of keratin fibres.

When they are present in composition (B), the colouring agent(s) are present in composition (B) preferably in a total content ranging from 0.001% to 10% by weight, more preferentially ranging from 0.01% to 4% by weight, even more preferentially ranging from 0.1% to 1% by weight, relative to the total weight of composition (B).

### Oxidation Dyes

The oxidation dyes are generally chosen from one or more oxidation bases, optionally combined with one or more coupling agents (also known as couplers).

### Oxidation Bases

Composition (B) may optionally comprise one or more oxidation bases advantageously chosen from those conventionally used in the dyeing of keratin fibres.

By way of example, the oxidation bases are chosen from para-phenylenediamines, bis(phenyl)alkylenediamines, para-aminophenols, ortho-aminophenols and heterocyclic bases, and the corresponding addition salts.

The para-phenylenediamines which may be mentioned include, for example, para-phenylenediamine, para-toluenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-N,N-bis(β-hydroxyethyl)amino-2-methylaniline, 4-N,N-bis(β-hydroxyethyl)amino-2-chloroaniline, 2-β-hydroxyethyl-para-phenylenediamine, 2-methoxymethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylenediamine, 4-aminophenylpyrrolidine, 2-thienyl-para-phenylenediamine, 2-β-hydroxyethylamino-5-aminotoluene and 3-hydroxy-1-(4′-aminophenyl)pyrrolidine, and the corresponding addition salts with an acid.

Preference is in particular given, among the abovementioned para-phenylenediamines, to para-phenylenediamine, para-toluenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine and the corresponding addition salts with an acid.

The bis(phenyl)alkylenediamines which may be mentioned include, for example, N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylenediamine, N,N′-bis(ethyl)-N,N′-bis(4′-amino-3′-methylphenyl)ethylenediamine and 1,8-bis(2,5-diaminophenoxy)-3,6-dioxaoctane, and the corresponding addition salts.

The para-aminophenols which are mentioned include, for example, para-aminophenol, 4-amino-3-methylphenol, 4-amino-3-fluorophenol, 4-amino-3-chlorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol and the corresponding addition salts with an acid.

The ortho-aminophenols which may be mentioned include, for example, 2-aminophenol, 2-amino-5-methylphenol, 2-amino-6-methylphenol and 5-acetamido-2-aminophenol and the corresponding addition salts.

Among the heterocyclic bases that may be mentioned, for example, are pyridine, pyrimidine and pyrazole derivatives.

The pyridine derivatives which may be mentioned include the compounds for example described in patents GB 1 026 978 and GB 1 153 196, for example 2,5-diaminopyridine, 2-(4-methoxyphenyl)amino-3-aminopyridine and 3,4-diaminopyridine and the corresponding addition salts.

Other pyridine oxidation bases that are useful in the present invention are the 3-aminopyrazolo[1,5-a]pyridine oxidation bases or the corresponding addition salts described, for example, in patent application FR 2 801 308. Examples which may be mentioned include pyrazolo[1,5-a]pyrid-3-ylamine, 2-acetylaminopyrazolo[1,5-a]pyrid-3-ylamine, 2-(morpholin-4-yl)pyrazolo[1,5-a]pyrid-3-ylamine, 3-aminopyrazolo[1,5-a]pyridine-2-carboxylic acid, 2-methoxypyrazolo[1,5-a]pyrid-3-ylamine, (3-aminopyrazolo[1,5-a]pyrid-7-yl)methanol, 2-(3-aminopyrazolo[1,5-a]pyrid-5-yl)ethanol, 2-(3-aminopyrazolo[1,5-a]pyrid-7-yl)ethanol, (3-aminopyrazolo[1,5-a]pyrid-2-yl)methanol, 3,6-diaminopyrazolo[1,5-a]pyridine, 3,4-diaminopyrazolo[1,5-a]pyridine, pyrazolo[1,5-a]pyridine-3,7-diamine, 7-(morpholin-4-yl)pyrazolo[1,5-a]pyrid-3-ylamine, pyrazolo[1,5-a]pyridine-3,5-diamine, 5-(morpholin-4-yl)pyrazolo[1,5-a]pyrid-3-ylamine, 2-[(3-aminopyrazolo[1,5-a]pyrid-5-yl)(2-hydroxyethyl)amino]ethanol, 2-[(3-aminopyrazolo[1,5-a]pyrid-7-yl)(2-hydroxyethyl)amino]ethanol, 3-aminopyrazolo[1,5-a]pyridin-5-ol, 3-aminopyrazolo[1,5-a]pyridin-4-ol, 3-aminopyrazolo[1,5-a]pyridin-6-ol, 3-aminopyrazolo[1,5-a]pyridin-7-ol, 2-β-hydroxyethoxy-3-aminopyrazolo[1,5-a]pyridine and 2-(4-dimethylpiperazinium-1-yl)-3-aminopyrazolo[1,5-a]pyridine, and the corresponding addition salts.

More particularly, the oxidation bases that are useful in the present invention are chosen from 3-aminopyrazolo[1,5-a]pyridines and are preferably substituted on carbon atom 2 with:
a) a (di)(C₁-C₆)(alkyl)amino group, it being possible for said alkyl group to be substituted by at least one hydroxyl, amino or imidazolium group;
b) an optionally cationic 5- to 7-membered heterocycloalkyl group containing from 1 to 3 heteroatoms, optionally substituted by one or more (C₁-C₆)alkyl groups, such as a di(C₁-C₄)alkylpiperazinium group; or
c) a (C₁-C₆)alkoxy group optionally substituted by one or more hydroxyl groups, such as a β-hydroxyalkoxy group, and the corresponding addition salts.

The pyrimidine derivatives which may be mentioned include the compounds described, for example, in patents DE 2359399, JP 88-169571, JP 05-63124 and EP 0770375 or patent application WO 96/15765, such as 2,4,5,6-tetraaminopyrimidine, 4-hydroxy-2,5,6-triaminopyrimidine, 2-hydroxy-4,5,6-triaminopyrimidine, 2,4-dihydroxy-5,6-diaminopyrimidine, 2,5,6-triaminopyrimidine and the addition salts thereof and the tautomeric forms thereof, when a tautomeric equilibrium exists.

The pyrazole derivatives which may be mentioned include the compounds described in patents DE 3843892 and DE 4133957 and patent applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethylpyrazole, 4,5-diamino-3-methyl-1-phenylpyrazole, 4,5-diamino-1-methyl-3-phenylpyrazole, 4-amino-1,3-dimethyl-5-hydrazinopyrazole, 1-benzyl-4,5-diamino-3-methylpyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropylpyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole, 3,5-diamino-4-(β-hydroxyethyl)amino-1-methylpyrazole and the corresponding addition salts. Use may also be made of 4,5-diamino-1-(β-methoxyethyl)pyrazole.

A 4,5-diaminopyrazole will preferably be used and more preferentially still 4,5-diamino-1-(β-hydroxyethyl)pyrazole and/or a corresponding salt.

The pyrazole derivatives which may also be mentioned comprise diamino-N,N-dihydropyrazolopyrazolones and in particular those described in patent application FR-A-2 886 136, such as the following compounds and the corresponding addition salts: 2,3-diamino-6,7-dihydro-1H,5H-pyrazolo[1,2-a]pyrazol-1-one, 2-amino-3-ethylamino-6,7-dihydro-1H,5H-pyrazolo[1,2-a]pyrazol-1-one, 2-amino-3-isopropylamino-6,7-dihydro-1H,5H-pyrazolo[1,2-a]pyrazol-1-one, 2-amino-3-(pyrrolidin-1-yl)-6,7-dihydro-1H,5H-pyrazolo[1,2-a]pyrazol-1-one, 4,5-diamino-1,2-dimethyl-1,2-dihydropyrazol-3-one, 4,5-diamino-1,2-diethyl-1,2-dihydropyrazol-3-one, 4,5-diamino-1,2-di(2-hydroxyethyl)-1,2-dihydropyrazol-3-one, 2-amino-3-(2-hydroxyethyl)amino-6,7-dihydro-1H,5H-pyrazolo[1,2-a]pyrazol-1-one, 2-amino-3-dimethylamino-6,7-dihydro-1H,5H-pyrazolo[1,2-a]pyrazol-1-one, 2,3-diamino-5,6,7,8-tetrahydro-1H,6H-pyridazino[1,2-a]pyrazol-1-one, 4-amino-1,2-diethyl-5-(pyrrolidin-1-yl)-1,2-dihydropyrazol-3-one, 4-amino-5-(3-dimethylaminopyrrolidin-1-yl)-1,2-diethyl-1,2-dihydropyrazol-3-one and 2,3-diamino-6-hydroxy-6,7-dihydro-1H,5H-pyrazolo[1,2-a]pyrazol-1-one.

Use will preferably be made of 2,3-diamino-6,7-dihydro-1H,5H-pyrazolo[1,2-a]pyrazol-1-one and/or a corresponding salt.

Use will preferably be made, as heterocyclic bases, of 4,5-diamino-1-(β-hydroxyethyl)pyrazole and/or 2,3-diamino-6,7-dihydro-1H,5H-pyrazolo[1,2-a]pyrazol-1-one and/or a corresponding salt.

### Coupling Agents

Composition (B) may optionally comprise one or more coupling agents advantageously chosen from those conventionally used in the dyeing of keratin fibres.

Mention may in particular be made, among these coupling agents, of meta-phenylenediamines, meta-aminophenols, meta-diphenols, naphthalene-based coupling agents and heterocyclic coupling agents, and also the corresponding addition salts. Mention may be made, for example, of 1,3-dihydroxybenzene, 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N-(β-hydroxyethyl)amino-3,4-methylenedioxybenzene, 2,6-bis(3-hydroxyethylamino)toluene, 6-hydroxyindoline, 2,6-dihydroxy-4-methylpyridine, 1H-3-methylpyrazol-5-one, 1-phenyl-3-methylpyrazol-5-one, 2,6-dimethylpyrazolo[1,5-b][1,2,4]triazole, 2,6-dimethyl[3,2-c][1,2,4]triazole, 6-methylpyrazolo[1,5-a]benzimidazole, 2-methyl-5-aminophenol, 5-N-(β-hydroxyethyl)amino-2-methylphenol, 3-aminophenol and 3-amino-2-chloro-6-methylphenol, the corresponding addition salts with an acid and the corresponding mixtures.

In general, the addition salts of oxidation bases and of coupling agents which can be used in the context of the invention are chosen in particular from the addition salts with an acid, such as the hydrochlorides, hydrobromides, sulfates, citrates, succinates, tartrates, lactates, tosylates, benzenesulfonates, phosphates and acetates.

The oxidation base(s) each advantageously represent from 0.001% to 10% by weight relative to the total weight of composition (B) and preferably from 0.005% to 5% by weight relative to the total weight of composition (B).

The coupling agent(s), if they are present, each advantageously represent(s) from 0.001% to 10% by weight relative to the total weight of composition (B), and preferably from 0.005% to 5% by weight relative to the total weight of composition (B).

### Direct Dyes

Composition (B) may also comprise one or more direct dyes.

The synthetic direct dyes which can be used are, for example, chosen from those conventionally used for direct dyeing, and among which mention may be made of all the aromatic and/or non-aromatic dyes which are commonly used, such as nitrobenzene, azo, hydrazono, nitro(hetero)aryl, tri(hetero)arylmethane, (poly)methine, carbonyl, azine, porphyrin, metalloporphyrin, quinone and in particular anthraquinone, indoamine and phthalocyanine direct dyes, and mixtures thereof.

Mention may be made, among the nitrobenzene direct dyes, of: 1,4-diamino-2-nitrobenzene, 1-amino-2-nitro-4-β-hydroxyethylaminobenzene, 1-amino-2-nitro-4-bis(β-hydroxyethyl)aminobenzene, 1,4-bis(β-hydroxyethylamino)-2-nitrobenzene, 1-β-hydroxyethylamino-2-nitro-4-bis(β-hydroxyethylamino)benzene, 1-β-hydroxyethylamino-2-nitro-4-aminobenzene, 1-β-hydroxyethylamino-2-nitro-4-(ethyl)(β-hydroxyethyl)aminobenzene, 1-amino-3-methyl-4-β-hydroxyethylamino-6-nitrobenzene, 1-amino-2-nitro-4-β-hydroxyethylamino-5-chlorobenzene, 1,2-diamino-4-nitrobenzene, 1-amino-2-β-hydroxyethylamino-5-nitrobenzene, 1,2-bis(β-hydroxyethylamino)-4-nitrobenzene, 1-amino-2-tris(hydroxymethyl)methylamino-5-nitrobenzene, 1-hydroxy-2-amino-5-nitrobenzene, 1-hydroxy-2-amino-4-nitrobenzene, 1-hydroxy-3-nitro-4-aminobenzene, 1-hydroxy-2-amino-4,6-dinitrobenzene, 1-β-hydroxyethyloxy-2-β-hydroxyethylamino-5-nitrobenzene, 1-methoxy-2-β-hydroxyethylamino-5-nitrobenzene, 1-β-hydroxyethyloxy-3-methylamino-4-nitrobenzene, 1-β,γ-dihydroxypropyloxy-3-methylamino-4-nitrobenzene, 1-β-hydroxyethylamino-4-β,γ-dihydroxypropyloxy-2-nitrobenzene, 1-β,γ-dihydroxypropylamino-4-trifluoromethyl-2-nitrobenzene, 1-β-hydroxyethylamino-4-trifluoromethyl-2-nitrobenzene, 1-β-hydroxyethylamino-3-methyl-2-nitrobenzene, 1-β-aminoethylamino-5-methoxy-2-nitrobenzene, 1-hydroxy-2-chloro-6-ethylamino-4-nitrobenzene, 1-hydroxy-2-chloro-6-amino-4-nitrobenzene, 1-hydroxy-6-bis(β-hydroxyethyl)amino-3-nitrobenzene, 1-β-hydroxyethylamino-2-nitrobenzene, 1-hydroxy-4-β-hydroxyethylamino-3-nitrobenzene.

Mention may be made, among the azo direct dyes, of: Basic Red 51, Basic Orange 31, Disperse Red 17, Acid Yellow 9, Acid Black 1, Basic Red 22, Basic Red 76, Basic Yellow 57, Acid Yellow 36, Acid Orange 7, Acid Red 33, Acid Red 35, Acid Yellow 23, Acid Orange 24, Disperse Black 9, Basic Brown 16, Basic Brown 17.

Mention may be made, among the hydrazono direct dyes, of: Basic Yellow 87.

Mention may be made, among the nitroaryl direct dyes, of: HC Blue 2, HC Yellow 2, HC Red 3, 4-hydroxypropylamino-3-nitrophenol, N,N′-bis(2-hydroxyethyl)-2-nitrophenylenediamine.

Mention may be made, among the triarylmethane direct dyes, of: Basic Violet 1, Basic Violet 2, Basic Violet 3, Basic Violet 4, Basic Violet 14, Basic Blue 1, Basic Blue 7, Basic Blue 26, Basic Green 1, Basic Blue 77 (also known as HC Blue 15), Acid Blue 1; Acid Blue 3; Acid Blue 7, Acid Blue 9; Acid Violet 49; Acid Green 3; Acid Green 5; Acid Green 50.

Mention may be made, among the quinone direct dyes, of: Disperse Red 15, Solvent Violet 13, Acid Violet 43, Disperse Violet 1, Disperse Violet 4, Disperse Blue 1, Disperse Violet 8, Disperse Blue 3, Disperse Red 11, Acid Blue 62, Disperse Blue 7, Basic Blue 22, Disperse Violet 15, Basic Blue 99, and also the following compounds: 1-N-methylmorpholiniumpropylamino-4-hydroxyanthraquinone, 1-aminopropylamino-4-methylaminoanthraquinone, 1-aminopropylaminoanthraquinone, 5-β-hydroxyethyl-1,4-diaminoanthraquinone, 2-aminoethylaminoanthraquinone, 1,4-bis(β,γ-dihydroxypropylamino)anthraquinone, Acid Blue 25, Acid Blue 43, Acid Blue 78, Acid Blue 129, Acid Blue 138, Acid Blue 140, Acid Blue 251, Acid Green 25, Acid Green 41, Acid Violet 42, Mordant Red 3, Acid Black 48, HC Blue 16.

Mention may be made, among the azine direct dyes, of: Basic Blue 17, Basic Red 2.

Mention may be made, among the indoamine direct dyes, of: 2-β-hydroxyethlyamino-5-[bis(β-4′-hydroxyethyl)amino]anilino-1,4-benzoquinone, 2-β-hydroxyethylamino-5-(2′-methoxy-4′-amino)anilino-1,4-benzoquinone, 3-N-(2′-chloro-4′-hydroxy)phenylacetylamino-6-methoxy-1,4-benzoquinoneimine, 3-N-(3′-chloro-4′-methylamino)phenylureido-6-methyl-1,4-benzoquinoneimine, 3-[4′-N-(ethylcarbamylmethyl)amino]phenylureido-6-methyl-1,4-benzoquinoneimine.

The natural direct dyes which can be used are, for example, chosen from lawsone, juglone, indigo, leucoindigo, indirubin, isatin, hennotannic acid, alizarin, carthamin, morin, purpurin, carminic acid, kermesic acid, laccaic acid, purpurogallin, protocatechualdehyde, curcumin, spinulosin, apigenidin, orceins, carotenoids, betanin, chlorophylls, chlorophyllins, monascus, polyphenols or ortho-diphenols.

Mention may be made, among the ortho-diphenols of use according to the invention, of: catechin, quercetin, brazilin, haematein, haematoxylin, chlorogenic acid, caffeic acid, gallic acid, L-DOPA, cyanidin, (−)-epicatechin, (−)-epigallocatechin, (−)-epigallocatechin 3-gallate (EGCG), isoquercetin, pomiferin, esculetin, 6,7-dihydroxy-3-(3-hydroxy-2,4-dimethoxyphenyl)coumarin, santalin A and B, mangiferin, butein, maritimetin, sulfuretin, robtein, betanidin, pericampylinone A, theaflavin, proanthocyanidin A2, proanthocyanidin B2, proanthocyanidin C1, procyanidins DP 4-8, tannic acid, purpurogallin, 5,6-dihydroxy-2-methyl-1,4-naphthoquinone, alizarin, wedelolactone and the natural extracts containing them.

The direct dye(s) may be present in composition (B) in a total content ranging from 0.001% to 5% by weight, preferably from 0.01% to 3% by weight, more preferentially from 0.1% to 1% by weight, even more preferentially from 0.1% to 0.8% by weight, relative to the total weight of composition (B).

According to a preferred embodiment, composition (B) comprises a total content of colouring agents of less than 0.1% by weight, preferably of less than 0.01% by weight, more preferentially of less than 0.001% by weight, relative to the total weight of composition (B).

According to a more preferred embodiment, composition (B) is free of colouring agents.

### pH of Composition (B)

Composition (B) preferably has a pH of less than or equal to 11, preferably less than or equal to 10.5, preferably less than or equal to 10.

The pH of composition (B) may vary from 8 to 11, preferably from 8 to 10.5, more preferentially from 8 to 10.

According to a particularly preferred embodiment, the pH of composition (B) according to the invention varies from 8.3 to 10.

Composition (B) may further comprise one or more compounds D as defined above. The various characteristics of the compounds D described above apply also to the compounds D which may be included in composition (B).

The compound(s) D are present in composition (B) preferably in a total content by weight ranging from 1% to 20% by weight, more preferentially ranging from 2% to 15% by weight, even more preferentially ranging from 2% to 12% by weight, relative to the total weight of composition (B).

Compositions (A), (B) and/or (C)

### Basifying Agents

The compositions (A) and/or (B) and/or (C) may further comprise one or more basifying agents other than the carbonates, bicarbonates and silicates as defined above.

The basifying agent(s) may be mineral or organic. They may be chosen from i) aqueous ammonia, ii) alkanolamines such as monoethanolamine, diethanolamine, triethanolamine and derivatives thereof, iii) oxyethylenated and/or oxypropylenated ethylenediamines, iv) mineral or organic hydroxides, v) amino acids, preferably basic amino acids such as arginine, lysine, ornithine, citrulline and histidine, and vi) the compounds of formula (II) below:

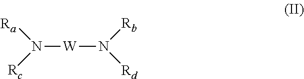

in which:
W is a divalent (C₁-C₈)alkylene group, preferably propylene, optionally substituted notably with a hydroxyl group or a C₁-C₄ alkyl radical;
Rₐ, R_{b}, R_{c} and R_{d}, which may be identical or different, represent a hydrogen atom or a C₁-C₄ alkyl or C₁-C₄ hydroxyalkyl radical;

vii) and mixtures thereof.

The mineral or organic hydroxides are preferably chosen from i) alkali metal hydroxides such as sodium hydroxide or potassium hydroxide, ii) alkaline-earth metal hydroxides, iii) transition metal hydroxides, such as hydroxides of metals from groups III, IV, V and VI, iv) hydroxides of lanthanides or of actinides.

When they are present, the basifying agents other than the carbonates, bicarbonates and silicates as defined above represent preferably from 0.001% to 20% by weight, more particularly from 0.005% to 16% relative to the total weight of composition (A) or (B) or (C).

According to a preferred embodiment, compositions (A) and/or (B) and/or (C) do not comprise a basifying agent chosen from aqueous ammonia and/or alkanolamines.

### Acidifying Agents

Compositions (A) and/or (B) and/or (C) may further comprise one or more acidifying agents.

Among the acidifying agents, examples that may be mentioned include mineral acids, for instance hydrochloric acid, (ortho)phosphoric acid, boric acid, nitric acid or sulfuric acid, or organic acids, for instance compounds comprising at least one carboxylic acid function such as acetic acid, tartaric acid, citric acid or lactic acid, a sulfonic acid function, a phosphonic acid function or a phosphoric acid function.

### Sequestrants

Compositions (A) and/or (B) and/or (C) may optionally comprise one or more sequestrants. As examples of sequestrants that may be used in the present invention, mention may be made of N,N-bis(carboxymethyl)-L-glutamic acid and tetrasodium N,N-bis(carboxymethyl)-L-glutamate.

Other Characteristics of Compositions (A), (B) and/or (C)

Compositions (A) and/or (B) and/or (C) preferably comprise water in a content ranging from 5% to 99% by weight, more preferentially ranging from 5% to 80% by weight relative to the total weight of the composition.

Compositions (A) and/or (B) and/or (C) may further comprise at least one organic solvent.

The term “organic solvent” means an organic substance which is capable of dissolving another substance without chemically modifying it.

Mention may be made, as organic solvent, for example, of lower C₂-C₄ alkanols, such as ethanol and isopropanol; polyols and polyol ethers, such as 2-butoxyethanol, propylene glycol, propylene glycol monomethyl ether, diethylene glycol monoethyl ether or diethylene glycol monomethyl ether, and also aromatic alcohols, such as benzyl alcohol or phenoxyethanol, and mixtures thereof.

The organic solvents are present in proportions ranging preferably from 0.1% to 40% by weight, more preferentially from 1% to 30% by weight, even more preferentially from 1% to 25% by weight, relative to the total weight of composition (A) and/or (B) and/or (C).

Compositions (A) and/or (B) and/or (C) may be provided in liquid form, in the form of a serum, in thickened form, in particular a gel, a cream, a wax or a paste, or in foam form. Compositions (A) and/or (B) and/or (C) may further comprise one or more additional compounds chosen from nonionic, anionic, cationic or amphoteric surfactants, cationic, anionic, nonionic or zwitterionic, associative or non-associative, thickening polymers of natural or synthetic origin, silicones in the form of oils, gums or resins or non-silicone plant, mineral or synthetic oils, UV-screening agents, fillers, such as pearlescent agents and metal oxides, such as titanium dioxides, clays, fragrances, peptizing agents, vitamins and preservatives.

### Additional Features of the Treatment Process

According to a preferred embodiment, the process according to the present invention is a process for lightening keratin fibres.

Preferably, the keratin fibres treated according to the process of the present invention are dark keratin fibres.

The term “dark keratin fibres” means keratin fibres with a tone depth (TD) of less than or equal to 6 (dark blond) and preferably less than or equal to 4 (chestnut-brown).

The process according to the present invention makes it possible to obtain a lightening of keratin fibres characterized by a value of b* which is lower, preferably 10% lower, more preferentially 15% lower, than the value of b* measured at the same intensity level L* on keratin fibres that have been lightened using a composition comprising one or more persulfates, the values of b* and L* being measured in the CIE L*a*b* system.

Preferably, the values of b* and L* are measured according to the colour evaluation method described in the examples.

### Composition (A)

Composition (A) is applied to dry or wet keratin fibres, preferably to dry keratin fibres.

Composition (A) is applied to the keratin fibres in an amount preferably ranging from 0.1 g to 20 g, more preferentially ranging from 0.5 g to 10 g of composition (A) per gram of keratin fibres.

Composition (A) is left on the keratin fibres for a time preferably ranging from 30 seconds to 30 minutes. By way of example, composition (A) is left on the keratin fibres for 5 minutes.

The application temperature of composition (A) is conventionally between room temperature (between 15° C. and 25° C.) and 70° C.

According to a preferred embodiment, composition (A) is applied at room temperature (between 15° C. and 25° C.).

Composition (A) can be left on the fibres under an occlusive system. A non-limiting example of an occlusive system that may be mentioned is an occlusive system such as aluminium wrap or plastic film or hair cap with or without holes.

After applying composition (A), the keratin fibres are optionally rinsed with water or optionally washed with a shampoo and then rinsed with water, before being optionally dried or left to dry naturally.

The drying step may be performed using absorbent paper, a hairdryer or a styling hood.

Preferably, the keratin fibres are neither rinsed with water, nor washed with shampoo after application of composition (A).

Preferably, the keratin fibres are dried or left to dry naturally after application of composition (A).

During the implementation of the process according to the present invention, the application of composition (A) can be repeated several times.

### Composition (B)

Composition (B) is applied to the keratin fibres in an amount preferably ranging from 0.1 g to 20 g of composition per gram of keratin fibres.

Composition (B) is left on the keratin fibres for a time preferably ranging from 1 minute to 1 hour, more preferentially ranging from 5 minutes to 60 minutes. By way of example, composition (B) is left on the keratin fibres for 50 minutes.

The application temperature of composition (B) is conventionally between room temperature (between 15° C. and 25° C.) and 80° C., preferably between room temperature and 60° C.

According to a preferred embodiment, composition (B) is applied at 27° C.

Composition (B) can be left on the fibres under an occlusive system. A non-limiting example of an occlusive system that may be mentioned is an occlusive system such as aluminium wrap or plastic film or hair cap with or without holes.

After applying composition (B), the keratin fibres are optionally rinsed with water or optionally washed with a shampoo and then rinsed with water, before being optionally dried or left to dry naturally.

The drying step may be performed using absorbent paper, a hairdryer or a styling hood.

Composition (B) used in the process according to the present invention is preferably prepared by mixing at least two compositions. Preferably, the mixing of said at least two compositions is carried out extemporaneously, before the application of the composition to the keratin fibres.

According to a preferred embodiment, composition (B) results from the mixing:
of a composition (B1) comprising:
   one or more chemical oxidizing agents chosen from hydrogen peroxide, hydrogen peroxide-generating systems other than peroxygenated salts and mixtures thereof as defined above, preferably hydrogen peroxide; and
of a composition (B2) comprising:
   one or more compounds chosen from carbonates, carbonate-generating systems, bicarbonates, bicarbonate-generating systems and mixtures thereof as defined above, preferably chosen from carbonates, bicarbonates and mixtures thereof as defined above; and
   one or more silicates as defined above; and
   optionally one or more colouring agents, preferably chosen from direct dyes, oxidation dyes and mixtures thereof; and
optionally of a composition (B3) comprising:
   one or more colouring agents, preferably chosen from direct dyes, oxidation dyes and mixtures thereof.

According to a preferred embodiment, the process according to the present invention is a process for lightening keratin fibres in which composition (B) results from the mixing:
of a composition (B1) comprising:
   one or more chemical oxidizing agents chosen from hydrogen peroxide, hydrogen peroxide-generating systems other than peroxygenated salts and mixtures thereof as defined above, preferably hydrogen peroxide; and
of a composition (B2) comprising:
   one or more compounds chosen from carbonates, carbonate-generating systems, bicarbonates, bicarbonate-generating systems and mixtures thereof as defined above, preferably chosen from carbonates, bicarbonates and mixtures thereof as defined above; and
   one or more silicates as defined above.

According to another preferred embodiment, the process according to the present invention is a process for simultaneous bleaching and dyeing of keratin fibres in which composition (B) results from the mixing:
of a composition (B1) comprising:
   one or more chemical oxidizing agents chosen from hydrogen peroxide, hydrogen peroxide-generating systems other than peroxygenated salts and mixtures thereof as defined above, preferably hydrogen peroxide; and
of a composition (B2) comprising:
   one or more compounds chosen from carbonates, carbonate-generating systems, bicarbonates, bicarbonate-generating systems and mixtures thereof as defined above, preferably chosen from carbonates, bicarbonates and mixtures thereof as defined above; and
   one or more silicates as defined above; and
   one or more colouring agents, preferably chosen from direct dyes, oxidation dyes and mixtures thereof.

According to another preferred embodiment, the process according to the present invention is a process for simultaneous bleaching and dyeing of keratin fibres in which composition (B) results from the mixing:
of a composition (B1) comprising:
   one or more chemical oxidizing agents chosen from hydrogen peroxide, hydrogen peroxide-generating systems other than peroxygenated salts and mixtures thereof as defined above, preferably hydrogen peroxide; and
of a composition (B2) comprising:
   one or more compounds chosen from carbonates, carbonate-generating systems, bicarbonates, bicarbonate-generating systems and mixtures thereof as defined above, preferably chosen from carbonates, bicarbonates and mixtures thereof as defined above; and
one or more silicates as defined above; and
   of a composition (B3) comprising one or more colouring agents, preferably chosen from direct dyes, oxidation dyes and mixtures thereof.

Preferably, at least one of the compositions (B1) or (B2) or at least one of the three compositions (B1) or (B2) or (B3) is aqueous. More preferentially, composition (B1) is aqueous.

According to a particular embodiment, the composition (B2) is anhydrous.

According to a particular embodiment, composition (B1) is aqueous, composition (B2) is anhydrous and composition (B3), when it is present, is aqueous.

An “aqueous composition” is understood to mean a composition comprising at least 2% by weight of water, preferably at least 5% by weight of water, more preferentially at least 10% by weight of water and even more advantageously more than 20% by weight of water.

An “anhydrous composition” is understood to mean a composition containing less than 2% by weight of water, preferably less than 0.5% by weight of water, more preferentially that is free of water. Where appropriate, such small amounts of water may notably be introduced by ingredients of the composition that may contain residual amounts thereof.

### Composition (C)

Composition (C) is applied to dry or wet keratin fibres, preferably to wet keratin fibres.

Composition (C) is applied to the keratin fibres in an amount preferably ranging from 0.01 g to 5 g, more preferentially ranging from 0.05 g to 5 g of composition (C) per gram of keratin fibres.

Composition (C) is left on the keratin fibres for a time preferably ranging from 30 seconds to 30 minutes, more preferentially ranging from 2 minutes to 10 minutes.

The application temperature of composition (C) is conventionally between room temperature (between 15° C. and 25° C.) and 70° C.

According to a preferred embodiment, composition (C) is applied at room temperature (between 15° C. and 25° C.).

Composition (C) can be left on the fibres under an occlusive system. A non-limiting example of an occlusive system that may be mentioned is an occlusive system such as aluminium wrap or plastic film or hair cap with or without holes.

According to a preferred embodiment, composition (C) is in the form of a shampoo. After applying composition (C), the keratin fibres are preferably rinsed with water and optionally treated using a composition of cream rinse, lotion or mask type, before being optionally dried or left to dry naturally.

According to another preferred embodiment, composition (C) is in the form of a hair conditioner composition, e.g. a cream rinse, a lotion or a mask. According to this other embodiment, the process comprises, between the step of applying composition (B) and the step of applying composition (C), a step of washing the keratin fibres with a shampoo, followed by rinsing with water. According to this other embodiment, after applying composition (C), the keratin fibres are preferably rinsed with water, before being optionally dried or left to dry naturally.

During the implementation of the process according to the present invention, the application of composition (C) can be repeated several times. By way of example, the application of composition (C) can be repeated from 1 to 5 times.

### Composition (B)

According to a second aspect, a subject of the present invention is a composition (B) as defined previously comprising one or more compounds D as defined previously.

### Uses

According to a third aspect, a subject of the present invention is the use of composition(s) (A) and/or (C) as defined previously for reducing the damage to keratin fibres treated by a process for treating keratin fibres using composition (B) as defined previously.

According to a preferred embodiment, a subject of the present invention is the use of composition(s) (A) and/or (C) as defined above for protecting and/or repairing the keratin fibres treated by a process for treating keratin fibres using composition (B) as defined above.

According to a more preferred embodiment, a subject of the present invention is the use of composition(s) (A) and/or (C) as defined above for reducing the breakage of the keratin fibres treated by a process for treating keratin fibres using composition (B) as defined above.

### Multicompartment Device (Kit)

According to a fourth aspect, a subject of the present invention is a multicompartment device comprising:
a second compartment containing a composition (B1) comprising:
   one or more chemical oxidizing agents chosen from hydrogen peroxide, hydrogen peroxide-generating systems other than peroxygenated salts, and mixtures thereof;
a second compartment containing a composition (B2) comprising:
   one or more compounds chosen from carbonates, carbonate-generating systems, bicarbonates, bicarbonate-generating systems and mixtures thereof; and
   one or more silicates; and
   optionally one or more colouring agents, preferably chosen from direct dyes, oxidation dyes and their mixtures; and
a third compartment containing a composition (A) or (C) as defined above, it being possible for composition (A) or (C) to optionally comprise one or more colouring agents, preferably chosen from direct dyes, oxidation dyes and mixtures thereof; and
optionally a fourth compartment containing a composition (B3) comprising one or more colouring agents, preferably chosen from direct dyes, oxidation dyes and mixtures thereof;

it being understood that at least one of compositions (B1), (B2), (B3), (A) or (C) comprises one or more compounds D as defined previously.

According to a particular embodiment, the multicompartment device (kit) comprises:
a second compartment containing a composition (B1) comprising:
   one or more chemical oxidizing agents chosen from hydrogen peroxide, hydrogen peroxide-generating systems other than peroxygenated salts, and mixtures thereof;
a second compartment containing a composition (B2) comprising:
   one or more compounds chosen from carbonates, carbonate-generating systems, bicarbonates, bicarbonate-generating systems and mixtures thereof; and
   one or more silicates; and
   optionally one or more colouring agents, preferably chosen from direct dyes, oxidation dyes and mixtures thereof; and
a third compartment containing a composition (A) as defined above, it being possible for composition (A) to optionally comprise one or more colouring agents, preferably chosen from direct dyes, oxidation dyes and mixtures thereof; and
a fourth compartment containing a composition (C) as defined above, it being possible for composition (C) to optionally comprise one or more colouring agents, preferably chosen from direct dyes, oxidation dyes and mixtures thereof;
optionally a fifth compartment containing a composition (B3) comprising one or more colouring agents, preferably chosen from direct dyes, oxidation dyes and mixtures thereof;

it being understood that at least one of the compositions (B1), (B2), (B3), (A) or (C) comprises one or more compounds D as defined previously.

### EXAMPLES

The examples that follow allow the invention to be understood more clearly, without, however, being limiting in nature. In the examples which follow, unless otherwise indicated, all the amounts are shown as weight percentages relative to the total weight of the composition.

### Examples 1 to 3: Process for Treating Keratin Fibres with Pretreatment Step

The following pretreatment compositions A1 and A2 and also the bleaching compositions Bₓ and B_{y} were prepared and then applied according to the application protocol described below and evaluated according to the evaluation protocols described below:

### Pretreatment Compositions

TABLE 1

| Ingredients | A1 | A2 |
|---|---|---|
| L-Carnitine mandelate | 10 | — |
| (Compound 7′) | | |
| L-Carnitine tartrate | — | 15 |
| (Compound 4′) | | |
| Sodium hydroxide | qs pH = 8 | qs pH = 9.4 |
| Water | qs 100 | qs 100 |

### Bleaching Compositions

TABLE 2

| Ingredients | Bₓ | B_{y} |
|---|---|---|
| Oxidizing cream | 66.6 (i.e. | 66.6 (i.e. |
| Blond Studio 40 Vol. | 8.0 of | 8.0 of |
| L'Oréal Professionnel | H₂O₂) | H₂O₂) |
| (12% of H₂O₂) | | |
| Sodium silicate | 9.7 | 9.7 |
| CAS: [1344-09-8] | | |
| Ammonium bicarbonate | 10 | 10 |
| L-Carnitine mandelate | — | 3.7 |
| (Compound 7′) | | |
| Water | qs 100 | qs 100 |

### Application Protocol

### Step a): Pretreatment

Compositions A1 and A2 were applied to 3 locks of type IV and HT4 Brazilian hair, in the bath ratios (g of composition/g of hair) indicated in the table below. The hair was massaged with the fingers, making circular movements along the entire length of the lock (5 times), and the locks were then left for 5 minutes. The locks were wrung out and then dried with a hairdryer.

### Step b): Bleaching

The bleaching composition Bₓ or B_{y} was then applied to 3 locks of hair treated during step a) in the bath ratio (g of composition/g of hair) indicated in the table below. The locks were then placed on a hot plate at 27° C. (temperature representative of the temperature of the hair on the head) for 25 minutes then turned over and left for another 25 minutes on the hot plate. The locks were then rinsed with tap water for 30 seconds, wrung out, shampooed with a DOP shampoo and then dried at 60° C.

The locks were then evaluated in terms of colour and integrity according to the evaluation protocols described below.

### Bath Ratios

TABLE 3

| | Step a) | | Step b) | |
|---|---|---|---|---|
| | | Bath ratio | | Bath ratio |
| | | of the | | of the |
| | Pretreatment | pretreatment | Bleaching | bleaching |
| Example | composition | composition | composition | composition |
| 1 | A1 | 10 | Bₓ | 10 |
| 2 | A2 | 3 | Bₓ | 10 |
| 3 | A1 | 5 | B_{y} | 10 |

### Colour Evaluation Method

The colour of the locks was evaluated in the CIE L*a*b* system, using a Minolta Spectrophotometer CM3610A colorimeter (illuminant D65).

In this L* a* b* system, L* represents the intensity of the colour, a* indicates the shade of the colour on the green/red colour axis and b* indicates the shade of colour on the blue/yellow colour axis. The higher the value of L*, the lighter the colour. The higher the value of a*, the redder the colour and the higher the value of b*, the yellower the colour.

### Method for Evaluating the Integrity of the Hair

This test consists in pulling on wet hair to see if the hair breaks, is irreversibly deformed or is deformed then returns to its initial shape and length. The test is commonly used by hairstylists to evaluate the integrity of their clients' hair. The test consists in taking 6 hair fibres, soaking them in water for 30 seconds then grasping them in the hands, moving the fingers and thumbs of each hand apart so that between the hands there are 3 centimetres of fibres and then pulling so that the fibres are deformed by an additional 1 cm. The fibres are held for 3 seconds then the tension is gently released. The fibres are placed horizontally on white paper for 5 minutes to dry and are then evaluated.

In the laboratory, a ruler is used to ensure that the distances are respected. The test is repeated 5 times, i.e. a total of 30 fibres.

The integrity performance of the hair is classified by the following rules:

If the sum of the broken and irreversibly deformed fibres is equal to or greater than 10, the hair has a very poor integrity.

If the sum of the broken and irreversibly deformed fibres is between 6 and 9 inclusive, the hair has a poor integrity.

If the sum of the broken and irreversibly deformed fibres is between 2 and 5 inclusive, the hair has a good integrity.

If the sum of the broken and irreversibly deformed fibres is 1 or less, the hair has a very good integrity.

### Colorimetric Measurements

The results of the colorimetric measurements are summarized in the following table:

TABLE 4

| Example | L* | a* | b* |
|---|---|---|---|
| 1 | 40.6 | 9.5 | 22.8 |
| 2 | 41.1 | 8.3 | 21.7 |
| 3 | 40.8 | 9.0 | 21.7 |

The process according to the invention makes it possible to obtain colour shades characterized by relatively low values of b* and therefore a less yellow and more natural result.

### Measurements of the Integrity of the Hair

The results of the integrity measurements are summarized in the following table:

TABLE 5

| | | Number of fibres | |
|---|---|---|---|
| | Number of fibres | which have been | Observations |
| | which have | irreversibly | regarding the |
| Example | broken | deformed | integrity |
| Natural hair | 0 | 0 | Very good |
| 1 | 0 | 2 | Good |
| 2 | 0 | 2 | Good |
| 3 | 0 | 1 | Very good |

The process according to the invention is respectful of the integrity of the fibre and makes it possible to protect the hair, notably from breakage or from deformation.

### Examples 4 to 7: Process for Treating Keratin Fibres with Post-Treatment Step

The following post-treatment compositions C1 to C4 and also the bleaching compositions Bₓ and B_{y} described in Examples 1 to 3 were prepared and then applied according to the application protocol described below and evaluated according to the evaluation protocols described in Examples 1 to 3:

### Post-Treatment Compositions

TABLE 6

| Ingredients | C1 | C2 | C3 |
|---|---|---|---|
| L-Carnitine | 10 | — | — |
| mandelate | | | |
| (Compound 7′) | | | |
| L-Carnitine tartrate | — | 5 | — |
| (Compound 4′) | | | |
| L-Carnitine | — | — | 10 |
| phenylacetate | | | |
| (Compound 10′) | | | |
| Sodium hydroxide | qs pH = 4.2 | qs pH = 4.0 | qs pH = 3.9 |
| or | | | |
| hydrochloric acid | | | |
| Water | qs 100 | qs 100 | qs 100 |

TABLE 7

| Ingredients | C4 |
|---|---|
| Propylene glycol | 60 |
| Cetyltrimethylammonium | 2.4 |
| chloride | |
| Behenyltrimethylammonium | 0.75 |
| chloride | |
| Mixture of caprylic/capric acid | 0.9 |
| triglycerides | |
| Myristyl alcohol | 2 |
| L-Carnitine mandelate | 5 |
| (Compound 7′) | |
| Ethanol | qs 100 |

### Application Protocol

### Step b): Bleaching

The bleaching composition Bₓ or B_{y} was applied to 4 locks of HT3 natural brown Japanese hair in the bath ratio (g of composition/g of hair) indicated in the table below. The locks were then placed on a hot plate at 27° C. (temperature representative of the temperature of the hair on the head) for 25 minutes then turned over and left for another 25 minutes on the hot plate.

The locks were rinsed with tap water for 30 seconds, wrung out, shampooed with DOP shampoo and then wrung dry with a towel.

### Step c): Post-Treatment

Compositions C1 to C4 were then applied to the 4 locks treated during step b) in the bath ratio (g of composition/g of hair) indicated in the table below. The hair was massaged with the fingers, making circular movements along the entire length of the lock (5 times), and the locks were then left for 5 minutes. The locks were then rinsed with tap water for 30 seconds, wrung out and then dried at 60° C.

The application of compositions C3 and C4 was repeated a further 4 times in order to reach a total of 5 applications of compositions C3 and C4.

### Bath Ratios

TABLE 8

| | Step b) | | Step c) | |
|---|---|---|---|---|
| | | Bath ratio | | Bath ratio |
| | | of the | Post- | of the |
| | Bleaching | bleaching | treatment | post-treatment |
| Example | composition | composition | composition | composition |
| 4 | Bₓ | 10 | C1 | 2 |
| 5 | Bₓ | 10 | C2 | 1 |
| 6 | Bₓ | 10 | C3 | 0.5 |
| 7 | B_{y} | 10 | C4 | 2 |

### Colorimetric Measurements

The results of the colorimetric measurements are summarized in the following table:

TABLE 9

| Example | L* | a* | b* |
|---|---|---|---|
| 4 | 43.8 | 10.0 | 24.3 |
| 5 | 43.0 | 9.6 | 24.9 |
| 6 | 42.5 | 10.3 | 24.0 |
| 7 | 39.1 | 8.9 | 21.6 |

The process according to the invention makes it possible to obtain colour shades characterized by relatively low values of b* and therefore a less yellow and more natural result.

### Measurements of the Integrity of the Hair

The results of the integrity measurements are summarized in the following table:

TABLE 10

| | | Number of fibres | |
|---|---|---|---|
| | Number of fibres | which have been | Observations |
| | which have | irreversibly | regarding the |
| Example | broken | deformed | integrity |
| 4 | 0 | 0 | Very good |
| 5 | 0 | 1 | Very good |
| 6 | 0 | 1 | Very good |
| 7 | 0 | 1 | Very good |

The process according to the invention is respectful of the integrity of the fibre and makes it possible to protect the hair, notably from breakage or from deformation.

### Example 8: Process for Treating Keratin Fibres with Pretreatment Step and Post-Treatment Step

The pretreatment composition A1 described in Examples 1 to 3, the bleaching composition B_{y} described in Examples 1 to 3 and also the following post-treatment composition C5 were prepared then applied according to the application protocol described below and evaluated in terms of the integrity of the hair according to the evaluation protocol described in Examples 1 to 3:

### Post-Treatment Composition

TABLE 11

| Ingredients | C5 |
|---|---|
| Cetearyl alcohol | 6 |
| Myristyl stearate and myristyl | 1 |
| palmitate mixture | |
| Behentrimonium chloride | 3 |
| Amodimethicone (and) trideceth-6 | 1.22 |
| (and) cetrimonium chloride | |
| Citric Acid | 0.1 |
| Chlorhexidine digluconate | 0.2 |
| Phenoxyethanol | 0.5 |
| L-Carnitine mandelate | 5 |
| (Compound 7′) | |
| Potassium hydroxide | qs pH = 4 |
| Fragrance | 0.9 |
| Water | qs 100 |

### Step a): Pretreatment

Composition A1 was applied to a lock of type IV and HT4 Brazilian hair, in a bath ratio (g of composition/g of hair) equal to 1. The hair was massaged with the fingers, making circular movements along the entire length of the lock (5 times), and the lock was then left for 5 minutes. The lock was then wrung out and then dried with a hairdryer.

### Step b): Bleaching

The bleaching composition B_{y} was then applied to the lock of hair treated during step a) in a bath ratio (g of composition/g of hair) equal to 10. The lock was then placed on a hot plate at 27° C. (temperature representative of the temperature of the hair on the head) for 25 minutes then turned over and left for another 25 minutes on the hot plate. The lock was then rinsed with tap water for 30 seconds, wrung out, shampooed with a DOP shampoo and then wrung out.

### Step c): Post-Treatment

Composition C5 was then applied to the lock treated during step b) in a bath ratio (g of composition/g of hair) equal to 0.4. The hair was massaged with the fingers, making circular movements along the entire length of the lock (5 times), and the locks were then left for 5 minutes. The lock was then rinsed with tap water for 30 seconds, wrung out and then dried at 60° C.

### Measurements of the Integrity of the Hair

The results of the integrity measurements are summarized in the following table:

TABLE 12

| | | Number of fibres | |
|---|---|---|---|
| | Number of fibres | which have been | Observations |
| | which have | irreversibly | regarding the |
| Example | broken | deformed | integrity |
| 8 | 0 | 0 | Very good |

The process according to the invention is respectful of the integrity of the fibre and makes it possible to protect the hair, notably from breakage or from deformation.

### Examples 9 to 11: Process for Treating Keratin Fibres without Pretreatment Step and Without Post-Treatment Step

The following bleaching compositions B_{z} and Bₜ were prepared.

Bleaching compositions B_{z}, Bₜ as well as bleaching composition B_{y} described in Examples 1 to 3 were applied according to the application protocol described below. Bleaching compositions B_{z} and Bₜ were evaluated in terms of the colour and integrity of the hair according to the evaluation protocols described in Examples 1 to 3. Bleaching composition B_{y} was evaluated in terms of the integrity of the hair according to the evaluation protocol described in Examples 1 to 3.

TABLE 13

| Ingredients | B_{z} | Bₜ |
|---|---|---|
| Oxidizing cream | 66.6 (i.e. | 66.6 (i.e. |
| Blond Studio 40 Vol. | 8.0 of | 8.0 of |
| L'Oréal Professionnel | H₂O₂) | H₂O₂) |
| (12% of H₂O₂) | | |
| Sodium Silicate | 9.7 | 9.7 |
| CAS: [1344-09-8] | | |
| Ammonium | 10 | 10 |
| Bicarbonate | | |
| L-carnitine tartrate | 2.0 | 5.0 |
| (Compound 1′) | | |
| Eau | Qsp 100 | Qsp 100 |

### Step b): Bleaching

The bleaching compositions B_{y}, B_{z} and Bₜ were applied to a lock of type IV and HT4 Brazilian hair, in a bath ratio (g of composition/g of hair) equal to 10. The locks were then placed on a hot plate at 27° C. (temperature representative of the temperature of the hair on the head) for 25 minutes then turned over and left for another 25 minutes on the hot plate. The locks were then rinsed with tap water for 30 seconds, wrung out, shampooed with a DOP shampoo, wrung out and then dried at 60° C.

### Colorimetric Measurements

The results of the colorimetric measurements are summarized in the following table:

TABLE 14

| | Bleaching | | | |
|---|---|---|---|---|
| Example | composition | L* | a* | b* |
| 9 | B_{z} | 43.55 | 9.56 | 22.95 |
| 10 | Bₜ | 44.84 | 9.51 | 22.39 |

The process according to the invention makes it possible to obtain colour shades characterized by relatively low values of b* and therefore a less yellow and more natural result.

### Measurements of the Integrity of the Hair

The results of the integrity measurements are summarized in the following table:

TABLE 15

| | | | Number of fibres | |
|---|---|---|---|---|
| | | Number of | which have been | Observations |
| | Bleaching | fibres which | irreversibly | regarding the |
| Example | composition | have broken | deformed | integrity |
| 9 | B_{z} | 2 | 1 | Good |
| 10 | Bₜ | 1 | 2 | Good |
| 11 | B_{y} | 0 | 2 | Very good |

The process according to the invention is respectful of the integrity of the fibre and makes it possible to protect the hair, notably from breakage or from deformation.

1-29. (canceled)

30. A method for treating keratin fibers, comprising:
a) applying to the keratin fibers at least one compound D chosen from compounds of formula (I), solvates thereof, or mixtures thereof: 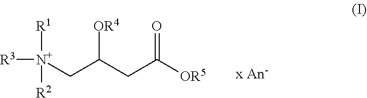
wherein in formula (I):
R¹, R² and R³ are independently chosen from a (hydroxy)(C₁-C₄)alkyl group;
R⁴ is chosen from a hydrogen atom, a (C₁-C₄)alkyl group, or a group —C(O)R, wherein R is a (C₁-C₃)alkyl group;
R⁵ is chosen from a hydrogen atom, a (C₁-C₄)alkyl group, an alkali metal, or an alkaline-earth metal;
An⁻ represents an organic anion;
x is a stoichiometric coefficient chosen to allow the electrical neutrality of the compound of formula (I);
b) applying to the keratin fibers a composition (B) comprising:
i) at least one chemical oxidizing agent chosen from hydrogen peroxide, hydrogen peroxide-generating systems other than peroxygenated salts, or mixtures thereof;
ii) at least one compound chosen from a carbonate, a carbonate-generating system, a bicarbonate, a bicarbonate-generating system, or mixtures thereof; and
iii) at least one silicate;
wherein the at least one compound (D) and composition (B) are applied simultaneously or sequentially to the keratin fibers.

31. The method of claim 30, wherein the at least one compound (D) is in composition (B).

32. The method of claim 30, wherein the at least one compound (D) is in a separate composition (A),
wherein composition (A) is applied to the keratin fibers before composition (B).

33. The method of claim 30, wherein the at least one compound (D) is in a separate composition (C),
wherein composition (C) is applied to the keratin fibers after composition (B).

34. The method of claim 30,
wherein the total amount of compound(s) D present in composition (A) ranges from 1% to 25% by weight, relative to the total weight of composition (A); and/or
wherein the total amount of compound(s) D present in composition (B) ranges from 1% to 20% by weight, relative to the total weight of composition (B); and/or
wherein the total amount of compound(s) D present in composition (C) ranges from 1% to 25% by weight, relative to the total weight of composition (C).

35. The method of claim 30, wherein An⁻ represents an organic anion chosen from:
a compounds of formula ⁻O—C(O)-ALK—C(O)—OM (IIa),
wherein:
ALK represents a (C₁-C₆)alkylene group optionally substituted with one or more hydroxyl (—OH) groups;
M represents a hydrogen atom, a negative charge, an alkali metal, or an alkaline-earth metal;
a compound of formula (IIb), (IIIb), or (IVb): 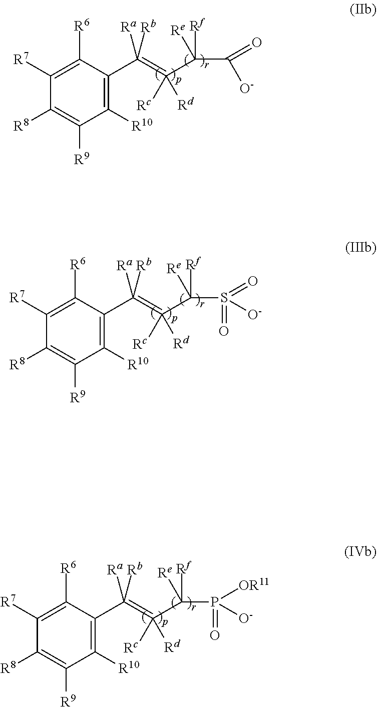
wherein:
R⁶ to R¹⁰ are independently chosen from:
a hydrogen atom;
a (C₁-C₆)alkyl or (C₁-C₆)alkoxy group, the (C₁-C₆)alkyl or (C₁-C₆)alkoxy group being optionally substituted with one or more identical or different groups chosen from: —OR, —NR′R″, —C(O)OR, —O—C(O)—R, aryl and —CF₃ and/or optionally interrupted with one or more groups or heteroatoms chosen from: O, NR′, CO, S, SO and SO₂; R representing a hydrogen atom or a (C₁-C₄)alkyl group, R′ and R″ representing, independently of each other, a hydrogen atom, a (C₁-C₄)alkyl group or a (C₁-C₄)alkylcarbonyl group wherein the group —NR′R″ is other than an —NH₂ group;
a halogen atom;
a group —O—X, in which X represents a hydrogen atom or a protecting group for the phenol function, wherein any two adjacent groups R6, R7, R8, R9 or R10 may form, together with the carbon atoms that bear them, a saturated, unsaturated or aromatic 5- or 6-membered heterocycle;
R^{a}, R^{b}, R^{c}, R^{d}, R^{e} and R^{f} are independently chosen from:
a hydrogen atom;
a (C₁-C₆)alkyl group optionally substituted with one or more identical or different groups chosen from: —OR, —NR′R″, —C(O)OR, —O—C(O)—R, (hetero)cycle, —C(O)—R, —NH—C(O)—R, —C(O)—NH—R, with R, R′ and R″, wherein the group —NR′R″ is other than an —NH₂ group;
a (C₁-C₆)alkoxy group optionally substituted with one or more identical or different groups chosen from: —OR, —NR′R″, —C(O)OR, —O—C(O)—R, aryl and —CF₃ and/or optionally interrupted with one or more groups or heteroatoms chosen from: O, NR′, CO, S, SO and SO₂, with R, R′ and R″, wherein the group —NR′R″ is other than an —NH₂ group;
a halogen atom;
a group —O—X with X as defined previously;
wherein:
(R^{a} and R^{b}) and/or (R^{c} and R^{d}) may form, together with the carbon atom which bears them, a C═O bond;
R^{a} and R^{b} may form, together with the carbon atom which bears them, a saturated or unsaturated, 4- to 8-membered (hetero)cycle;
R^{a} or R^{b} or R^{c} or R^{d} may form with R⁶ or R¹⁰ and the carbon atoms that bear them a 4- to 8-membered (hetero)cycle; [symbol]
represents a single bond or an ethylenic double bond, with the proviso that when [symbol] represents an ethylenic double bond, R^{b} and R^{c} are absent;
p is an integer equal to 0 or 1;
r is an integer equal to 0 or 1;
R¹¹ represents a hydrogen atom, a negative charge, an alkali metal or an alkaline-earth metal, preferably a negative charge; the compounds of formula (IIb), (IIIb) or (IVb); or
mixtures thereof.

36. The method of claim 30, wherein An⁻ represents an organic anion chosen from compounds of formulae 1 to 46:

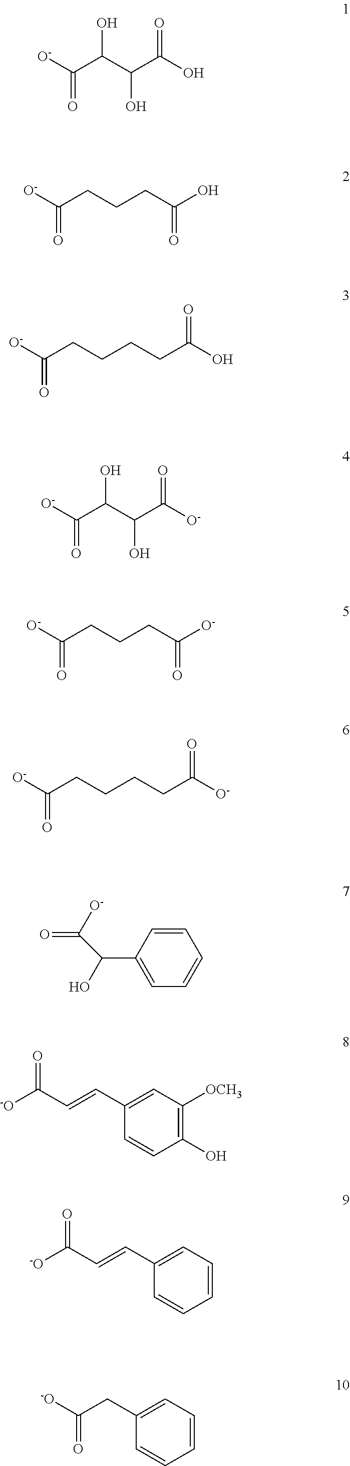

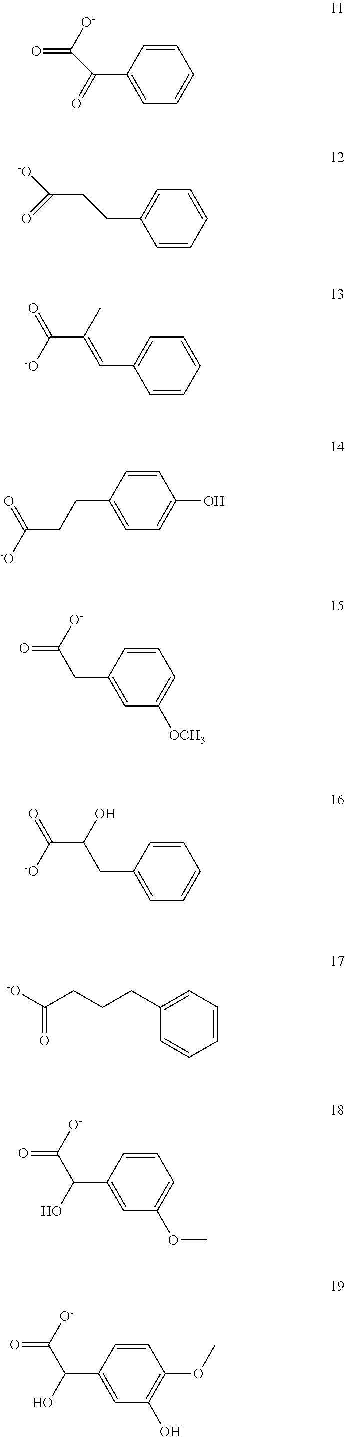

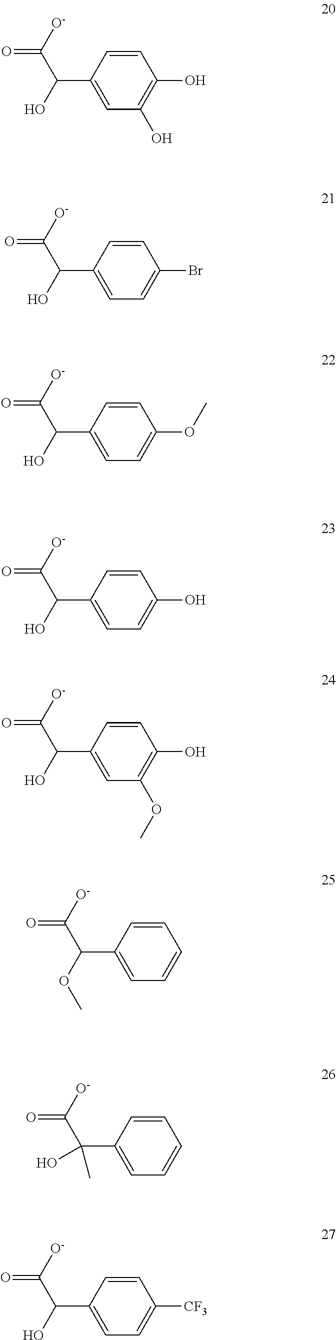

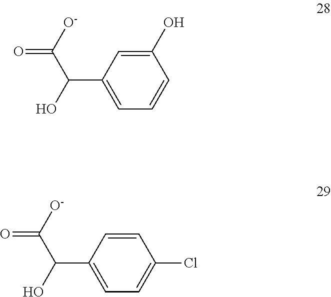 [SMILES: O=C([O-])C(O)c1cccc(O)c1 ; O=C([O-])C(O)c1ccc(Cl)cc1]

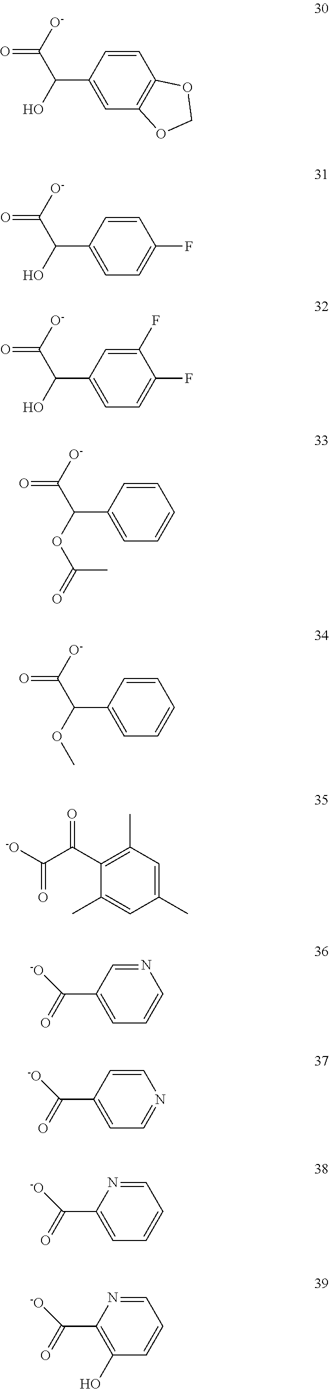

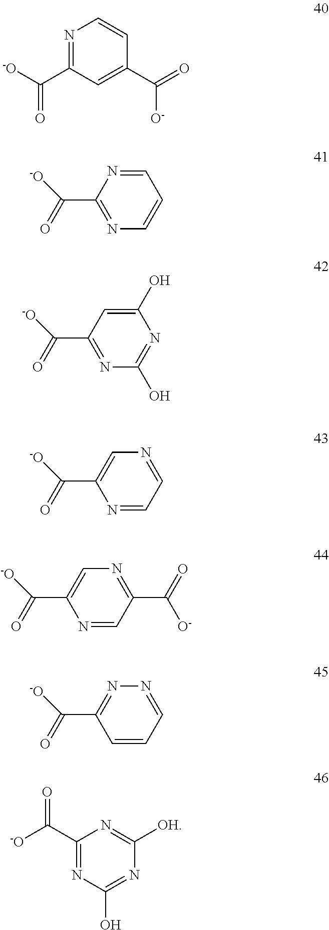

37. The method of claim 30, wherein:
R¹, R² and R³ represent a methyl group;
R⁴ and R⁵ represent a hydrogen atom.

38. The method of claim 30, wherein the compounds of formula (I) are chosen from compounds of formulae 1′ to 46′:

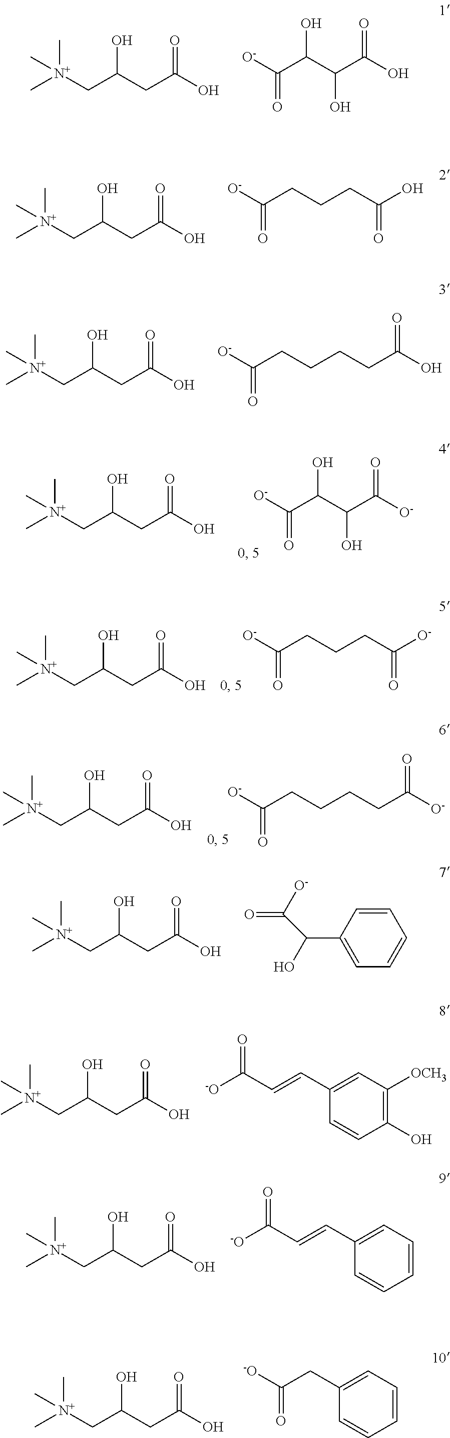

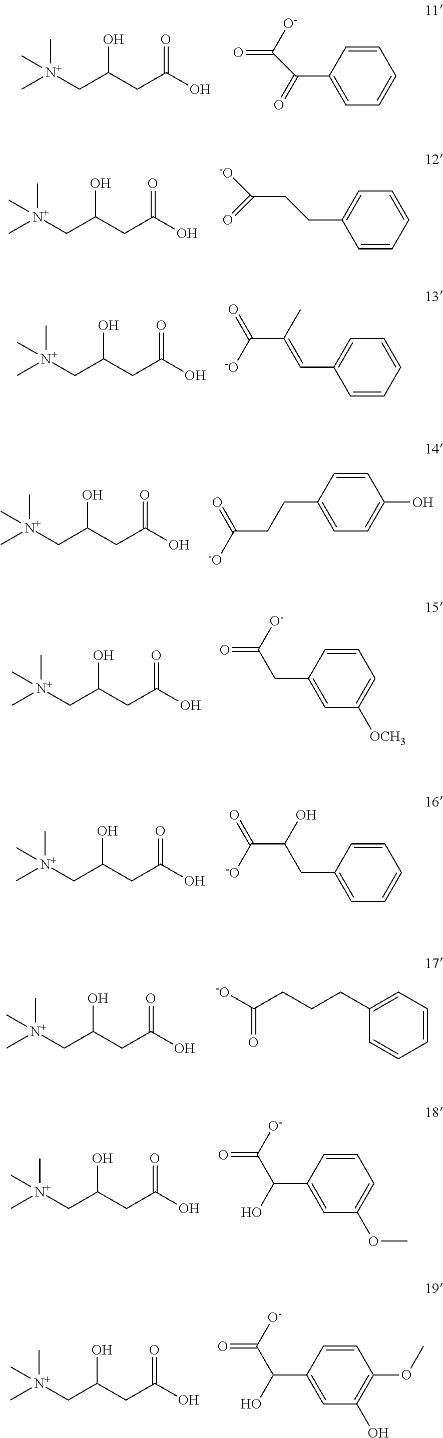

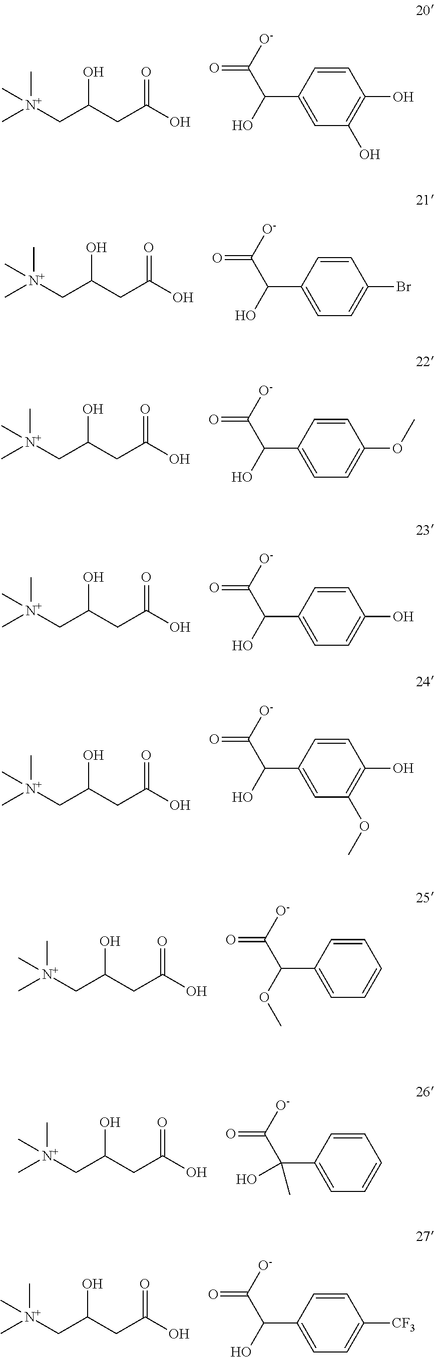

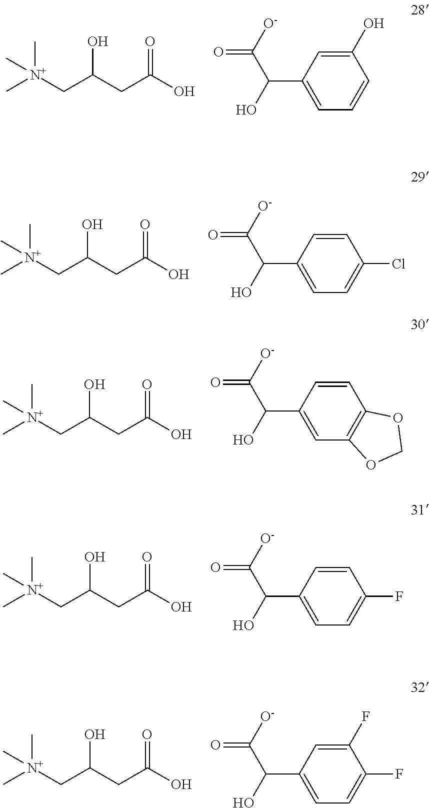

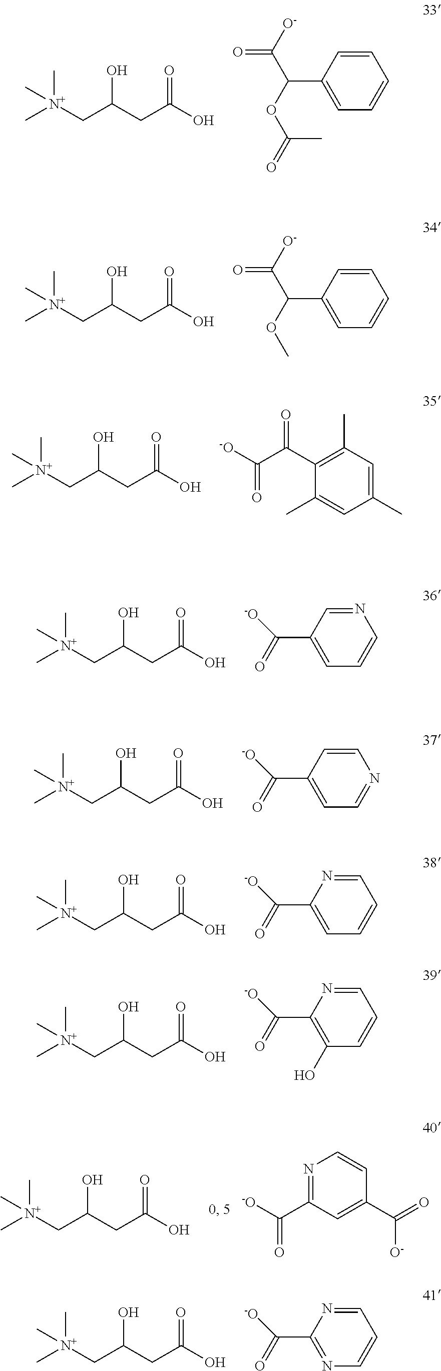

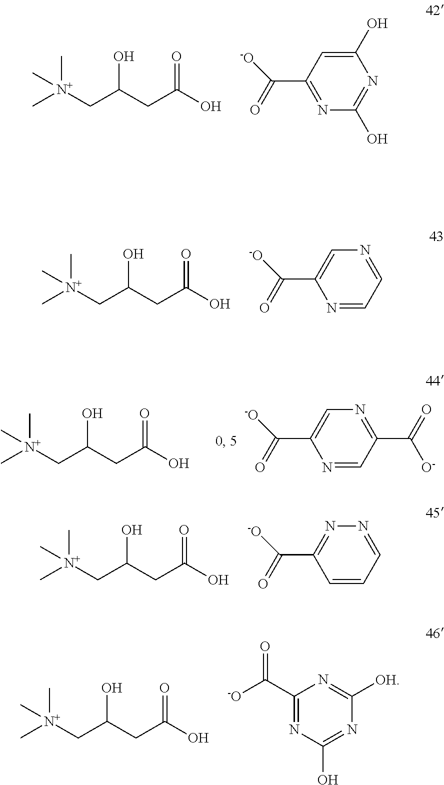

39. The method of claim 30, wherein the at least one chemical oxidizing agent in composition (B) is hydrogen peroxide.

40. The method of claim 30, wherein the total amount of chemical oxidizing agent(s) ranges from 1% to 12% by weight, relative to the total weight of composition (B).

41. The method of claim 30, wherein the total amount of compound(s) ii) ranges from 0.01% to 20% by weight, relative to the total weight of composition (B).

42. The method of claim 30, wherein the carbonate is chosen from:
an alkali metal carbonate;
an alkaline-earth metal carbonate;
a lanthanide carbonate;
a transition metal carbonate;
bismuth carbonate;
cadmium carbonate;
thallium carbonate;
zinc carbonate;
a compound of formula (N⁺R¹R²R³R⁴)₂CO₃²⁻ wherein R¹, R², R³, and R⁴ represent, independently of each other, a hydrogen atom or a (C₁-C₄)alkyl group optionally substituted with a hydroxyl group;
guanidine carbonate; or
mixtures thereof.

43. The method of claim 30, wherein the bicarbonate is chosen from:
an alkali metal bicarbonate;
an alkaline-earth metal bicarbonate;
a compound of formula N⁺R¹R²R³R⁴, ·HCO₃⁻ wherein R¹, R², R³, and R⁴ represent, independently of one another, a hydrogen atom or a (C₁-C₄)alkyl group optionally substituted by a hydroxyl group;
aminoguanidine bicarbonate; or
mixtures thereof.

44. The method of claim 30, wherein the at least one silicate iii) is chosen from an alkali metal silicate, an alkaline-earth metal silicate, aluminium silicate, trimethylammonium silicate, or mixtures thereof.

45. The method of claim 30, wherein the total amount of silicate(s) iii) present in composition (B) ranges from 1% to 40% by weight, relative to the total weight of composition (B).

46. The method of claim 30, wherein composition (B) comprises a total amount of persulfates of less than 10% by weight, relative to the total weight of composition (B).

47. The method of claim 30, wherein composition (B) further comprises iv) at least one coloring agent chosen from direct dyes, oxidation dyes, or mixtures thereof.

48. The method of claim 30, wherein composition (B) is a mixture of a composition (B1) with a composition (B2) and optionally a composition (B3):
wherein composition (B1) comprises:
the at least one chemical oxidizing agent i) chosen from hydrogen peroxide, hydrogen peroxide-generating systems other than peroxygenated salts, or mixtures thereof; and
wherein composition (B2) comprises:
the at least one compound ii) chosen from a carbonate, a carbonate-generating system, a bicarbonate, a bicarbonate-generating system, or mixtures thereof;
the at least one silicate iii); and
optionally at least one coloring agent iv); and
wherein composition (B3) comprises the at least one coloring agent iv).

49. A multi-compartment device comprising:
a first compartment comprising a composition (B1), wherein composition (B1) comprises at least one chemical oxidizing agent i) chosen from hydrogen peroxide, hydrogen peroxide-generating systems other than peroxygenated salts, or mixtures thereof;
a second compartment comprising a composition (B2), wherein composition (B2) comprises:
at least one compound ii) chosen from a carbonate, a carbonate-generating system, a bicarbonate, a bicarbonate-generating system, or mixtures thereof;
at least one silicate iii); and
optionally at least one coloring agent iv); and
optionally a third compartment comprising a composition (B3), wherein composition (B3) comprises the at least one coloring agent iv); and
optionally a fourth compartment comprising a composition (A); and
optionally a fifth compartment comprising a composition (C);
wherein at least one of composition (B1), composition (B2), composition (B3), composition (A) or composition (C) comprises at least one compound D chosen from compounds of formula (I), solvates thereof, or mixtures thereof: 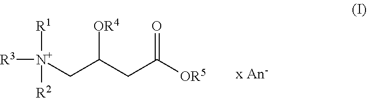
wherein in formula (I):
R¹, R² and R³ are independently chosen from a (hydroxy)(C₁-C₄)alkyl group;
R⁴ is chosen from a hydrogen atom, a (C₁-C₄)alkyl group, or a group —C(O)R, wherein R is a (C₁-C₃)alkyl group;
R⁵ is chosen from a hydrogen atom, a (C₁-C₄)alkyl group, an alkali metal, or an alkaline-earth metal;
An⁻ represents an organic anion;
x is a stoichiometric coefficient chosen to allow the electrical neutrality of the compound of formula (I).
